SEQUENCE LISTING (47 sequences)

SEQ ID NO: 1 - DNA, 1227 bp, Parascedosporium putredinis
atgccttctgcgaagcgtcttctcgggctcctgctcgccgccaccgcggcggtcggtgtagctgcccaggaacctgccctcaccgaggatgatttcagcatccccgagatcgagggaggtgatgccctcgcacagcttgcccagctcgcggcggattcttctcaggagactgccctcaggatggcgaagcgcggtctgaacagcggctgcagccctagccagatcaaggttagaagggaatggcgaaccctgacttctgctcagcgcaagcagtacattgcgtcggtcaagtgtcttcagacgaagcccagcttcttcgaccccaacatcatcccggccgctaagtcgctatttgacgactttgttggtgtccatgttttccagactgggtccatccatctcactgccactttcctcacatggcatcgctacttcgtctatacctacgagacgaagctccgcgaggaatgcggttacactggacccttgccctactgggagtggggactagacgtcaacaaccccaacgcctccccggtcttcgacggctccgacacgtctctgagcggcaacggtgccttctttgcgcatgagggcatccagatggtgcagcctatcaacggcaacatcctcaagctcccccccggcaacggtggtggctgcgtgaccaagggtcccttcaaggacatgaaggttcactttggcaccatcatcctgcccgtgtacggccagcctatcctcagtggtgtcgagaaccccattgccgacaacgagcgctgcctcaagcgcgatctcaatgccggcatcgctaagcgcttcactagcttcctcaactcgaccagcgtgattctcaagaacaacaacatcgagatgttccaggcccatctgcagggcgacgaccgctacgtgctcaaccagctcggtgttcacggtggtggtcactacaccatcggcggtgaccccggtggtgatcccttcatctcccctggtgacccggctttctacctccaccacgcccagattgaccgcatctactggatctggcagatgctcgacttcaagaaccgtcagggtgtccacggtaccgccaccctccagaacaaccctcccagcgccaacgttaccgttgaggacaccatcgacctctctcccctcgccccgcctgtcaagatcaaggacctcatgaacactgtcggaggctcgcccttgtgctacatctacctctaa

SEQ ID NO: 2 - DNA, 1158 bp, Parascedosporium putredinis
atgtccgtcgtcaagaagctcctcgccgccctcgcggccaccactttcctcaccggcgtcgctgctcagacctatgagtttagcgaggaggagctcacctctggtgatgccctcaaggcccttagcaagcaggctatggaaaatgctcttgcccgcctccccgaatctggagagggctgcacgcgggagaacgtcaaaattcgaaaggaatggcgcaacatgccggccgagatgagaatcggctatgtcagcgctctccagtgcttgatggaatccgaaagcgaatatcccgacgtcgacggtgccaagacggcgtttgacgacttcgccgttcttcattacaacctcacgccgttcgtgcataactctgctaccttccttaccttccacaggtactacattcacaccctggaagagcagatgaggaacaagtgcggatacactggtgacttcccctactgggagtggggcctcgactgcgacgacccgcaacagtctcccctctttgacggctccgaaacctctctcggcagcgacggtgagcctgtggaggccggagccggcggtggcttcggcggcggcttcggctttggcatgggaggcggcagcggtggtggctgcgtgatgaagggacccttctccaactacaccgtcaacctcggaccctcaaccaccgccgacccgctcgcatataacccgcgctgcatcaagcgaaacctgaacggcgctatctgcaagcagaatgcctcgctccggaacacgacgacgacgatccttgactcgcccgatatcgaactattccaggcgattgtccaaggtgacatgcgataccccgaggcgaggggtcttggcatggccgtccacggcggtggtcactttactattggtggtgatcccggtggcgacttctacttctcccctctcgagcccgccttcttccaacaccacggccagatcgaccgcatgtactttgtctggcagaacctcgactgggaaacccggcagaacattgccggcaccggtaccatgatgaaccagccccccagcccagaggttgagatcaccgaactcctcgacctcagcccccttgcagaagccaggccgatcaaggacctcattgacacccttggctcggccccgttctgctttgtttacgag

SEQ ID NO: 3 - DNA, 1230 bp, Parascedosporium putredinis
atgctcgtgtacgcttcgctggcgatcctgccccttcttgccggggtgggcgcatcgccgcttaacaagaaggccaccttttcgtaccagcaggtatctcgaaatcctgacttcccgttggatgtggtcgacgagctcgaggccaaagccatgccgggggttgagacatggatggctgggaaggacaacgccaacggctgcacgcttgaaaatgctgcggtccgtagagaatggggtgacttatccgttgccgagcgtgaagagtacgttgccgcggttctctgtctccagaagttaccatccaaggcgcccgagggaaaggcaccaggtgcactcagccgcttcgatgacttcgttgccacgcacatgacacaggccatgatgcttcactcgcccaccaatctgtttgcgagccaccgttactatatctgggcctacgaaaccgctcttcgtgaagagtgcggctatacgggctaccagccgtacatgaattacgaccgttatgccgatgacctcctcaactcccccttgttcaacgggaacgcgtccagcctaggaggcaacggagctccaagccaatacgccggtgtccctcagccattcaggcctccgtacaacatgatcccctctgctggaggcggtggttgtgtgaccgagggtccattcaaagacatggtcgtgagtcttggccccgtgggaatcatcgtcaatgatatccccaagaacccgcgctccgatggcctcggctccaacccgcgatgcctccgtcgggacgtgaataaattttcggcggccggcgccaaggccaactacacgtacaacctgattacggagaacgccggaattgatgcgttttataaccgctacctcgggcagccgcagctgaaggatgatcccaatccttgggggctccataatgccggccactatatcgtaggcggcgacccaggcggtgatttctacgcttcccccggcgacccctacttctacttccaccacggcatgttggaccgcgtgtggtggatctggcagatgcaggacccggccgcccgcgtcaaccttattccgggtaccggcgccccggcgatgaaccatcccggcatgccgatgaaccgtcggcagtcgtccgcgacaattgtggacttgggttggacggcgccggcggtgcccattacggagctcaacgattccttgggcggtaacggcggcaagttttgctacgtgtacgtg

SEQ ID NO: 4 - DNA, 1152 bp, Parascedosporium putredinis
atgcgtctgttcaaggctctcgccgccgcggccctgaccggcctcgtcgcggccgatgctgtcaatgacctagagaccaagggccgtgccgccctcgacgccgtcattgagagctctaccacgtgtagcaaggacaagctcaaggtccgcagagagtggggagatatcagcaccaccgagcgcaaggcttacctcgacggagtgctgtgcctcctgaacacaccctccaagctcgatcccgctcgttaccccggcgccaagaaccgctacgatgactttgtcgttgttcacatgaaccagaccctctccatccatggaaccggtaacttcctcgtgtggcaccgctactacgtgtgggcctgggagaacgtcatgaggaccgagtgtggctatgaaggaacccagccctactgggactacggccgctgggccgaggatcctctttcctcgcctctcttcgacggaagcgagacttcgcttggcggaaacggcgcccccgtaacccagaacaagcgcagccgcgtggaaggccgccagttcggcggtggcggtggtttcggtggtggccttggcggaggtttcggaggcggcggtgatggcggtggctgcatttccaccggtcccttcaaggacatggtcgtcaccctcggccccatgtccgccgtcgtcaggcccgcaccggcccgcaacccccaggccgacggctacggtagtaacccccgctgcatccgccgcgacatcaccaactcgctgagcatggcctacggaaagaccgaggacatcgtcaacagcatcgtcaactacaacgacatccttgccttccagaacttcatgcagggcggtaccggcgtgcacggcgtcggccacttcaccgtctccggcgaccccggtggtgatttctacatctcccccaacgagccttccttctggctccaccacgccatgattgaccgcatctggaccatctggcagtcccaggactacgagacccgcaggggcgccatggagggaggcaccagcatgatgggaggtggcagggcccagtccctcgatgacctcgtcgacctcggcgtcattgccgacactgtctaccccatccgcgacatcctcagctctgttgacggccccggccccttctgctacgtgtacgag

SEQ ID NO: 5 - DNA, 1185 bp, Parascedosporium putredinis
atgcgttctgctttggctctcgtactcgcggcctctctcctcggcggagaggccagcagcatcaagaagcgattctcgacactcgatgtttggcgccacggcgactacgagcgggatatcgtcgatcagctctccgacgaaacgtttcccaagatcgccgagtgggtcgagaagaccggctcgacctgcactctcgaaaatgccgtgcagcgaaaggagtggaccgatttgaccattgatgagagggcggactacatccaagcggtgcagtgcttgatgaagctccctcccaagtcgcaggaccaagttcccggctccctcaaccgatatgacgacttcgtggccactcacgttactggtattccggttcttcacgcacctaccaacctcttcgccagccacaggtactatatctgggcgtacgagttggcacttcgcgaggagtgcgggtacaagggctaccagccgtacatgaactacgagcgacaccaggatcccatcacctcgcccctgttcaacggaaatgccaccagcatgggcggcaatggagcggcggctgagtaccccggcgtagtcatgccttatcccaggccctacaacgtcattcccgctgcaggcggtggtggctgcgtcacggaaggtcccttctccgacatggttgtcagcatcggccctctgggcactgttctccgcgacattccccgcaacccccgcgccgatggcctcggttccaacccccgctgcctgcgacgcgatcttaacaagttctccgccgctggagcctccgccaaccactcgtactcgctcattatggactaccccgatgtcgacgccttctacaaccgatacctcggacagccgttcctgagaggagatgaattcccgtggggtcttcactctgccggtcactacatcacgggaggagacccaggtggcgacttttacgcctcgcctggtgacccgaccttctggatgcatcatgccgccctcgaccgcttgtggtggctgtggcagatgcaggatcccgagaaccgcctgcaggccatccccggcatcacttcgtcgaggatgaccaacgaggatgctcaaaagacaatggtggatctgaagtggacggcagagccccgctcgctcggagatctcaacgatcaaatgggaagtgcccccttctgttatatctatgta

SEQ ID NO: 6 - DNA, 1059 bp, Parascedosporium putredinis
atgcagctcaccattctcgcaacggcgcttcttgccgtcagcgcctccgcagcgcccacctgctgcaccaacgccgagcggcgagagtggagaactttctcaactaaggagaagcaagcctacatcgcggcagtgaaatgtcttcaaagcaagccatctcagttgaagagcacctacccgacgtcgcagaatcgattcgatgactttcaggcggttcatattgacctgacggagaagtatcactttactggacctttccaggcctggcatcgcgtctttctccacaagtacgaatccgacctccggggactttgtgcatacaagggctaccagccatactgggactggaccaaggattccgggtctgaggctgccttcctcgcctcgcctgtctttgacgctgttaatggcttcggtggaaacggaccttacgttgatacgtcgaactttcccgtcaccaacgtccccgtcaaaatcccgaataagactggcggaggctgtgttcaagatggcgcctttgtcaatatgacagtcactctcggcccaggccctagtcttgagtcgaacccccgctgtctcacccgtgacttcagctactggctcatctcacggaccctaaccaaggcggtggtagactggactcttgaggccgcgtctttcgctgtctttgacttccgtctgcagggcacaggcatcgagccagaaggcatgacagttcatgcaggtggccaccttggtgttggtggtgatatcggcgagatcggcaacatgtattcatcccccggagacccgctcttctatcttcaccacgccaatcttgacagactctgggaccagtggcagaggaagaagtttgctcagcgtgtccaggatatgaccggaccggatacaatgtgggcctatccattcaacttctttggcgacgtaccctataccaatatcactctggaaaccttgctcgacttcaagggtctccttgggtccagctcggcagaccgatatgtcaagatcaaggacgttatggacagccagggtccgaacctctgcgtcttctacaaa

SEQ ID NO: 7 - DNA, 1167 bp, Parascedosporium putredinis
atggtggcgatcagctacgtccttacggcgttggcggtggcgatccctgcccttgcgcagggtccgtgttccacaattcgtcaacgacgggcatggcatacgctgagcaacagcgagaaacgcgcttacttggatgccgaggtttgcttgctgggcaaggcccccaagtttggcttcgagggggccaaaaacaggttcgaggagctccaggccgctcatcaagtccaggcttatctcattcatggagttggagcgtttttgcctttccacagatatcttatgcacgctcatgagaccctgttgaggactgagtgtgggtaccagggagcgcaaccatactgggatgagacacgggatgccggtcgcgtcagtgagtccgagatccttgatccggacactggtttcggaggagatggtgtgggtgagaggggttgtatcgccgacggaccgtttgcaggctacatcaacagcatcgggccaggttataggattacggatcgctgcatcacacgcttcgtgaacaacacccgaagcttgatggctagcccccgtttcaccgacaggtgccaaggcatgaatcgatatgttgacgtgtggccatgcctcgagggaaaccctcataattcgggccatggcgctattagcggactgatgatggacccgatcgccagccccggcgaccccatcttctaccttcatcacacatggctcgataagctgtggtgggagtggcaggccatggacctcccccgacgcctcaccgacattggcggccgtaacacccaggacggctccgaaggtttccccggtgcaccgcccaactctaacggtcccaacggcgcgaagcgacgatcccccgccgacggccccatcctcatcttccccggggacggaggaaacattgactgggacgagattgactgggaaaagattggcttccccggcctcggcggtggaaatggaggcccgatccagttgccgcccggcgttgacgttcccccggaagccctggaacctcccgaggatgccgagcctcaagagcccaggggtgacccgggcgatgtgaccacgttgaaccacgtcctgaagatgtttggcctcgttccagacgccttgatccgagacgtcatggatatcgccggaggcactctgtgttacgagtacgtc

SEQ ID NO: 8 - DNA, 1044 bp, Parascedosporium putredinis
atgaaaaacctcgcgggactaataacggccttggccaccggcgttgggatggcgcatacgcatacacttgtgccgcacatgcaagactcaactccctgtatcaacccaagtttgcgacgtccatggcagattctctcggacggcgaaaaacgctcatatctcgatgcccagctttgtgtgatgagaacgccgcagaccctcggtcttcctggcgcgagaacacgtttcgaggagctggctgccacccaccagattggcgcccgtgccagccatgccacggggacatttttcccctaccatcgatacctactgcatgcccatgagtcattgctgaaggagtgcggctaccacgcaggtctcccttactgggatgagaccagggaagctggaaatttcatcaagtctaccatattcgaatcgggcctcggattcggtggctttggaagcgacctcaaagggtgcatcgaagacggacctttcgcaaacttgacaagtacaatcggtcccgggttttcgctgaacgaacactgcatctcacgtgcgctcaatgaaactgcagggctcaaggcggctagggaagaggttgataagtgcttagaagccaacgactatacagagatgtggcgttgtgcatataccacaccccatcgtgggggtcatgggggcgtgggaggcacgatgggagacgctttggcatcgcccggcgacccggtattctacgtccaccacgcttgggtcgataagatttggtgggattggcaggaggctgatcttgataatagaatgtatgctattggcgggcccagcttccagtcacctgatatcgggtttcctgaggttcctggtgatgttgaggaagaagaagcaaatatctttggcaaaccaagcgaagcaatccgacgactacaggagctatggagttcctctgatccaagcagagaaactaccctggagcataatctgacattgctaggtattattcccgacatcaacattagtaaggtcatggacactagaggtgggtatctttgctacgaatatgtt

SEQ ID NO: 9 - protein, 408 aa, Parascedosporium putredinis
MPSAKRLLGLLLAATAAVGVAAQEPALTEDDFSIPEIEGGDALAQLAQLAADSSQETALRMAKRGLNSGCSPSQIKVRREWRTLTSAQRKQYIASVKCLQTKPSFFDPNIIPAAKSLFDDFVGVHVFQTGSIHLTATFLTWHRYFVYTYETKLREECGYTGPLPYWEWGLDVNNPNASPVFDGSDTSLSGNGAFFAHEGIQMVQPINGNILKLPPGNGGGCVTKGPFKDMKVHFGTIILPVYGQPILSGVENPIADNERCLKRDLNAGIAKRFTSFLNSTSVILKNNNIEMFQAHLQGDDRYVLNQLGVHGGGHYTIGGDPGGDPFISPGDPAFYLHHAQIDRIYWIWQMLDFKNRQGVHGTATLQNNPPSANVTVEDTIDLSPLAPPVKIKDLMNTVGGSPLCYIYL

SEQ ID NO: 10 - protein, 386 aa, Parascedosporium putredinis
MSVVKKLLAALAATTFLTGVAAQTYEFSEEELTSGDALKALSKQAMENALARLPESGEGCTRENVKIRKEWRNMPAEMRIGYVSALQCLMESESEYPDVDGAKTAFDDFAVLHYNLTPFVHNSATFLTFHRYYIHTLEEQMRNKCGYTGDFPYWEWGLDCDDPQQSPLFDGSETSLGSDGEPVEAGAGGGFGGGFGFGMGGGSGGGCVMKGPFSNYTVNLGPSTTADPLAYNPRCIKRNLNGAICKQNASLRNTTTTILDSPDIELFQAIVQGDMRYPEARGLGMAVHGGGHFTIGGDPGGDFYFSPLEPAFFQHHGQIDRMYFVWQNLDWETRQNIAGTGTMMNQPPSPEVEITELLDLSPLAEARPIKDLIDTLGSAPFCFVYE

SEQ ID NO: 11 - protein, 410 aa, Parascedosporium putredinis
MLVYASLAILPLLAGVGASPLNKKATFSYQQVSRNPDFPLDVVDELEAKAMPGVETWMAGKDNANGCTLENAAVRREWGDLSVAEREEYVAAVLCLQKLPSKAPEGKAPGALSRFDDFVATHMTQAMMLHSPTNLFASHRYYIWAYETALREECGYTGYQPYMNYDRYADDLLNSPLFNGNASSLGGNGAPSQYAGVPQPFRPPYNMIPSAGGGGCVTEGPFKDMVVSLGPVGIIVNDIPKNPRSDGLGSNPRCLRRDVNKFSAAGAKANYTYNLITENAGIDAFYNRYLGQPQLKDDPNPWGLHNAGHYIVGGDPGGDFYASPGDPYFYFHHGMLDRVWWIWQMQDPAARVNLIPGTGAPAMNHPGMPMNRRQSSATIVDLGWTAPAVPITELNDSLGGNGGKFCYVYV

SEQ ID NO: 12 - protein, 384 aa, Parascedosporium putredinis
MRLFKALAAAALTGLVAADAVNDLETKGRAALDAVIESSTTCSKDKLKVRREWGDISTTERKAYLDGVLCLLNTPSKLDPARYPGAKNRYDDFVVVHMNQTLSIHGTGNFLVWHRYYVWAWENVMRTECGYEGTQPYWDYGRWAEDPLSSPLFDGSETSLGGNGAPVTQNKRSRVEGRQFGGGGGFGGGLGGGFGGGGDGGGCISTGPFKDMVVTLGPMSAVVRPAPARNPQADGYGSNPRCIRRDITNSLSMAYGKTEDIVNSIVNYNDILAFQNFMQGGTGVHGVGHFTVSGDPGGDFYISPNEPSFWLHHAMIDRIWTIWQSQDYETRRGAMEGGTSMMGGGRAQSLDDLVDLGVIADTVYPIRDILSSVDGPGPFCYVYE

SEQ ID NO: 13 - protein, 395 aa, Parascedosporium putredinis
MRSALALVLAASLLGGEASSIKKRFSTLDVWRHGDYERDIVDQLSDETFPKIAEWVEKTGSTCTLENAVQRKEWTDLTIDERADYIQAVQCLMKLPPKSQDQVPGSLNRYDDFVATHVTGIPVLHAPTNLFASHRYYIWAYELALREECGYKGYQPYMNYERHQDPITSPLFNGNATSMGGNGAAAEYPGVVMPYPRPYNVIPAAGGGGCVTEGPFSDMVVSIGPLGTVLRDIPRNPRADGLGSNPRCLRRDLNKFSAAGASANHSYSLIMDYPDVDAFYNRYLGQPFLRGDEFPWGLHSAGHYITGGDPGGDFYASPGDPTFWMHHAALDRLWWLWQMQDPENRLQAIPGITSSRMTNEDAQKTMVDLKWTAEPRSLGDLNDQMGSAPFCYIYV

SEQ ID NO: 14 - protein, 353 aa, Parascedosporium putredinis
MQLTILATALLAVSASAAPTCCTNAERREWRTFSTKEKQAYIAAVKCLQSKPSQLKSTYPTSQNRFDDFQAVHIDLTEKYHFTGPFQAWHRVFLHKYESDLRGLCAYKGYQPYWDWTKDSGSEAAFLASPVFDAVNGFGGNGPYVDTSNFPVTNVPVKIPNKTGGGCVQDGAFVNMTVTLGPGPSLESNPRCLTRDFSYWLISRTLTKAVVDWTLEAASFAVFDFRLQGTGIEPEGMTVHAGGHLGVGGDIGEIGNMYSSPGDPLFYLHHANLDRLWDQWQRKKFAQRVQDMTGPDTMWAYPFNFFGDVPYTNITLETLLDFKGLLGSSSADRYVKIKDVMDSQGPNLCVFYK

SEQ ID NO: 15 - protein, 389 aa, Parascedosporium putredinis
MVAISYVLTALAVAIPALAQGPCSTIRQRRAWHTLSNSEKRAYLDAEVCLLGKAPKFGFEGAKNRFEELQAAHQVQAYLIHGVGAFLPFHRYLMHAHETLLRTECGYQGAQPYWDETRDAGRVSESEILDPDTGFGGDGVGERGCIADGPFAGYINSIGPGYRITDRCITRFVNNTRSLMASPRFTDRCQGMNRYVDVWPCLEGNPHNSGHGAISGLMMDPIASPGDPIFYLHHTWLDKLWWEWQAMDLPRRLTDIGGRNTQDGSEGFPGAPPNSNGPNGAKRRSPADGPILIFPGDGGNIDWDEIDWEKIGFPGLGGGNGGPIQLPPGVDVPPEALEPPEDAEPQEPRGDPGDVTTLNHVLKMFGLVPDALIRDVMDIAGGTLCYEYV

SEQ ID NO: 16 - protein, 348 aa, Parascedosporium putredinis
MKNLAGLITALATGVGMAHTHTLVPHMQDSTPCINPSLRRPWQILSDGEKRSYLDAQLCVMRTPQTLGLPGARTRFEELAATHQIGARASHATGTFFPYHRYLLHAHESLLKECGYHAGLPYWDETREAGNFIKSTIFESGLGFGGFGSDLKGCIEDGPFANLTSTIGPGFSLNEHCISRALNETAGLKAAREEVDKCLEANDYTEMWRCAYTTPHRGGHGGVGGTMGDALASPGDPVFYVHHAWVDKIWWDWQEADLDNRMYAIGGPSFQSPDIGFPEVPGDVEEEEANIFGKPSEAIRRLQELWSSSDPSRETTLEHNLTLLGIIPDINISKVMDTRGGYLCYEYV

SEQ ID NO: 17 - DNA, 1158 bp, artificial sequence
  codon optimised sequence
caagaaccggcactgaccgaagatgattttagcattccggaaattgaaggtggtgatgcactggcacagctggcccaactggcagcagatagcagccaagaaaccgcactgcgtatggcaaaacgtggtctgaatagcggttgtagcccgagccagattaaagttcgtcgtgaatggcgtaccctgaccagcgcacagcgtaaacagtatatcgcaagcgttaaatgcctgcagaccaaaccgagcttttttgatccgaacattattccggcagcaaaaagcctgtttgatgattttgttggtgtgcatgtttttcagaccggcagcattcatctgaccgcaacctttctgacctggcatcgttattttgtgtatacctatgaaaccaaactgcgcgaagaatgtggttatacaggtccgctgccgtattgggaatggggtttagatgttaataatccgaatgccagtccggtttttgatggtagcgataccagcctgagcggtaatggtgcattttttgcacatgaaggtattcagatggtgcagccgattaatggcaatattctgaaactgcctcctggtaatggcggtggttgtgttaccaaaggtccgtttaaagatatgaaagtgcatttcggcaccattattctgccggtttatggccagccgattctgagcggtgttgaaaatccgattgcagataatgaacgttgcctgaaacgcgatctgaatgcaggtattgccaaacgttttaccagctttctgaatagtaccagcgtgattctgaagaacaacaacatcgaaatgtttcaggcccatctgcagggtgatgatcgttatgttctgaatcagctgggtgttcatggtggtggtcattataccattggtggtgatcctggtggcgatccgtttattagtccgggtgatccggcattttatctgcatcatgcacagattgatcgcatctattggatttggcagatgctggatttcaaaaatcgtcagggcgttcatggtacagcaacactgcagaataaccctccgagcgcaaatgttaccgttgaagataccattgatctgagtccgctggcaccgcctgttaaaatcaaagatctgatgaataccgttggtggcagtccgctgtgttatatctatctg

SEQ ID NO: 18 - DNA, 1161 bp, Paracedosporium putredinis
caggaacctgccctcaccgaggatgatttcagcatccccgagatcgagggaggtgatgccctcgcacagcttgcccagctcgcggcggattcttctcaggagactgccctcaggatggcgaagcgcggtctgaacagcggctgcagccctagccagatcaaggttagaagggaatggcgaaccctgacttctgctcagcgcaagcagtacattgcgtcggtcaagtgtcttcagacgaagcccagcttcttcgaccccaacatcatcccggccgctaagtcgctatttgacgactttgttggtgtccatgttttccagactgggtccatccatctcactgccactttcctcacatggcatcgctacttcgtctatacctacgagacgaagctccgcgaggaatgcggttacactggacccttgccctactgggagtggggactagacgtcaacaaccccaacgcctccccggtcttcgacggctccgacacgtctctgagcggcaacggtgccttctttgcgcatgagggcatccagatggtgcagcctatcaacggcaacatcctcaagctcccccccggcaacggtggtggctgcgtgaccaagggtcccttcaaggacatgaaggttcactttggcaccatcatcctgcccgtgtacggccagcctatcctcagtggtgtcgagaaccccattgccgacaacgagcgctgcctcaagcgcgatctcaatgccggcatcgctaagcgcttcactagcttcctcaactcgaccagcgtgattctcaagaacaacaacatcgagatgttccaggcccatctgcagggcgacgaccgctacgtgctcaaccagctcggtgttcacggtggtggtcactacaccatcggcggtgaccccggtggtgatcccttcatctcccctggtgacccggctttctacctccaccacgcccagattgaccgcatctactggatctggcagatgctcgacttcaagaaccgtcagggtgtccacggtaccgccaccctccagaacaaccctcccagcgccaacgttaccgttgaggacaccatcgacctctctcccctcgccccgcctgtcaagatcaaggacctcatgaacactgtcggaggctcgcccttgtgctacatctacctctaa

SEQ ID NO: 19 - DNA, 1092 bp, Paracedosporium putredinis
cagacctatgagtttagcgaggaggagctcacctctggtgatgccctcaaggcccttagcaagcaggctatggaaaatgctcttgcccgcctccccgaatctggagagggctgcacgcgggagaacgtcaaaattcgaaaggaatggcgcaacatgccggccgagatgagaatcggctatgtcagcgctctccagtgcttgatggaatccgaaagcgaatatcccgacgtcgacggtgccaagacggcgtttgacgacttcgccgttcttcattacaacctcacgccgttcgtgcataactctgctaccttccttaccttccacaggtactacattcacaccctggaagagcagatgaggaacaagtgcggatacactggtgacttcccctactgggagtggggcctcgactgcgacgacccgcaacagtctcccctctttgacggctccgaaacctctctcggcagcgacggtgagcctgtggaggccggagccggcggtggcttcggcggcggcttcggctttggcatgggaggcggcagcggtggtggctgcgtgatgaagggacccttctccaactacaccgtcaacctcggaccctcaaccaccgccgacccgctcgcatataacccgcgctgcatcaagcgaaacctgaacggcgctatctgcaagcagaatgcctcgctccggaacacgacgacgacgatccttgactcgcccgatatcgaactattccaggcgattgtccaaggtgacatgcgataccccgaggcgaggggtcttggcatggccgtccacggcggtggtcactttactattggtggtgatcccggtggcgacttctacttctcccctctcgagcccgccttcttccaacaccacggccagatcgaccgcatgtactttgtctggcagaacctcgactgggaaacccggcagaacattgccggcaccggtaccatgatgaaccagccccccagcccagaggttgagatcaccgaactcctcgacctcagcccccttgcagaagccaggccgatcaaggacctcattgacacccttggctcggccccgttctgctttgtttacgag

SEQ ID NO: 20 - DNA, 1176 bp, Paracedosporium putredinis
tcgccgcttaacaagaaggccaccttttcgtaccagcaggtatctcgaaatcctgacttcccgttggatgtggtcgacgagctcgaggccaaagccatgccgggggttgagacatggatggctgggaaggacaacgccaacggctgcacgcttgaaaatgctgcggtccgtagagaatggggtgacttatccgttgccgagcgtgaagagtacgttgccgcggttctctgtctccagaagttaccatccaaggcgcccgagggaaaggcaccaggtgcactcagccgcttcgatgacttcgttgccacgcacatgacacaggccatgatgcttcactcgcccaccaatctgtttgcgagccaccgttactatatctgggcctacgaaaccgctcttcgtgaagagtgcggctatacgggctaccagccgtacatgaattacgaccgttatgccgatgacctcctcaactcccccttgttcaacgggaacgcgtccagcctaggaggcaacggagctccaagccaatacgccggtgtccctcagccattcaggcctccgtacaacatgatcccctctgctggaggcggtggttgtgtgaccgagggtccattcaaagacatggtcgtgagtcttggccccgtgggaatcatcgtcaatgatatccccaagaacccgcgctccgatggcctcggctccaacccgcgatgcctccgtcgggacgtgaataaattttcggcggccggcgccaaggccaactacacgtacaacctgattacggagaacgccggaattgatgcgttttataaccgctacctcgggcagccgcagctgaaggatgatcccaatccttgggggctccataatgccggccactatatcgtaggcggcgacccaggcggtgatttctacgcttcccccggcgacccctacttctacttccaccacggcatgttggaccgcgtgtggtggatctggcagatgcaggacccggccgcccgcgtcaaccttattccgggtaccggcgccccggcgatgaaccatcccggcatgccgatgaaccgtcggcagtcgtccgcgacaattgtggacttgggttggacggcgccggcggtgcccattacggagctcaacgattccttgggcggtaacggcggcaagttttgctacgtgtacgtg

SEQ ID NO: 21 - DNA, 1098 bp, Paracedosporium putredinis
gatgctgtcaatgacctagagaccaagggccgtgccgccctcgacgccgtcattgagagctctaccacgtgtagcaaggacaagctcaaggtccgcagagagtggggagatatcagcaccaccgagcgcaaggcttacctcgacggagtgctgtgcctcctgaacacaccctccaagctcgatcccgctcgttaccccggcgccaagaaccgctacgatgactttgtcgttgttcacatgaaccagaccctctccatccatggaaccggtaacttcctcgtgtggcaccgctactacgtgtgggcctgggagaacgtcatgaggaccgagtgtggctatgaaggaacccagccctactgggactacggccgctgggccgaggatcctctttcctcgcctctcttcgacggaagcgagacttcgcttggcggaaacggcgcccccgtaacccagaacaagcgcagccgcgtggaaggccgccagttcggcggtggcggtggtttcggtggtggccttggcggaggtttcggaggcggcggtgatggcggtggctgcatttccaccggtcccttcaaggacatggtcgtcaccctcggccccatgtccgccgtcgtcaggcccgcaccggcccgcaacccccaggccgacggctacggtagtaacccccgctgcatccgccgcgacatcaccaactcgctgagcatggcctacggaaagaccgaggacatcgtcaacagcatcgtcaactacaacgacatccttgccttccagaacttcatgcagggcggtaccggcgtgcacggcgtcggccacttcaccgtctccggcgaccccggtggtgatttctacatctcccccaacgagccttccttctggctccaccacgccatgattgaccgcatctggaccatctggcagtcccaggactacgagacccgcaggggcgccatggagggaggcaccagcatgatgggaggtggcagggcccagtccctcgatgacctcgtcgacctcggcgtcattgccgacactgtctaccccatccgcgacatcctcagctctgttgacggccccggccccttctgctacgtgtacgag

SEQ ID NO: 22 - DNA, 1131 bp, Paracedosporium putredinis
agcagcatcaagaagcgattctcgacactcgatgtttggcgccacggcgactacgagcgggatatcgtcgatcagctctccgacgaaacgtttcccaagatcgccgagtgggtcgagaagaccggctcgacctgcactctcgaaaatgccgtgcagcgaaaggagtggaccgatttgaccattgatgagagggcggactacatccaagcggtgcagtgcttgatgaagctccctcccaagtcgcaggaccaagttcccggctccctcaaccgatatgacgacttcgtggccactcacgttactggtattccggttcttcacgcacctaccaacctcttcgccagccacaggtactatatctgggcgtacgagttggcacttcgcgaggagtgcgggtacaagggctaccagccgtacatgaactacgagcgacaccaggatcccatcacctcgcccctgttcaacggaaatgccaccagcatgggcggcaatggagcggcggctgagtaccccggcgtagtcatgccttatcccaggccctacaacgtcattcccgctgcaggcggtggtggctgcgtcacggaaggtcccttctccgacatggttgtcagcatcggccctctgggcactgttctccgcgacattccccgcaacccccgcgccgatggcctcggttccaacccccgctgcctgcgacgcgatcttaacaagttctccgccgctggagcctccgccaaccactcgtactcgctcattatggactaccccgatgtcgacgccttctacaaccgatacctcggacagccgttcctgagaggagatgaattcccgtggggtcttcactctgccggtcactacatcacgggaggagacccaggtggcgacttttacgcctcgcctggtgacccgaccttctggatgcatcatgccgccctcgaccgcttgtggtggctgtggcagatgcaggatcccgagaaccgcctgcaggccatccccggcatcacttcgtcgaggatgaccaacgaggatgctcaaaagacaatggtggatctgaagtggacggcagagccccgctcgctcggagatctcaacgatcaaatgggaagtgcccccttctgttatatctatgta

SEQ ID NO: 23 - DNA, 1008 bp, Paracedosporium putredinis
gcgcccacctgctgcaccaacgccgagcggcgagagtggagaactttctcaactaaggagaagcaagcctacatcgcggcagtgaaatgtcttcaaagcaagccatctcagttgaagagcacctacccgacgtcgcagaatcgattcgatgactttcaggcggttcatattgacctgacggagaagtatcactttactggacctttccaggcctggcatcgcgtctttctccacaagtacgaatccgacctccggggactttgtgcatacaagggctaccagccatactgggactggaccaaggattccgggtctgaggctgccttcctcgcctcgcctgtctttgacgctgttaatggcttcggtggaaacggaccttacgttgatacgtcgaactttcccgtcaccaacgtccccgtcaaaatcccgaataagactggcggaggctgtgttcaagatggcgcctttgtcaatatgacagtcactctcggcccaggccctagtcttgagtcgaacccccgctgtctcacccgtgacttcagctactggctcatctcacggaccctaaccaaggcggtggtagactggactcttgaggccgcgtctttcgctgtctttgacttccgtctgcagggcacaggcatcgagccagaaggcatgacagttcatgcaggtggccaccttggtgttggtggtgatatcggcgagatcggcaacatgtattcatcccccggagacccgctcttctatcttcaccacgccaatcttgacagactctgggaccagtggcagaggaagaagtttgctcagcgtgtccaggatatgaccggaccggatacaatgtgggcctatccattcaacttctttggcgacgtaccctataccaatatcactctggaaaccttgctcgacttcaagggtctccttgggtccagctcggcagaccgatatgtcaagatcaaggacgttatggacagccagggtccgaacctctgcgtcttctacaaa

SEQ ID NO: 24 - DNA, 1110 bp, Paracedosporium putredinis
cagggtccgtgttccacaattcgtcaacgacgggcatggcatacgctgagcaacagcgagaaacgcgcttacttggatgccgaggtttgcttgctgggcaaggcccccaagtttggcttcgagggggccaaaaacaggttcgaggagctccaggccgctcatcaagtccaggcttatctcattcatggagttggagcgtttttgcctttccacagatatcttatgcacgctcatgagaccctgttgaggactgagtgtgggtaccagggagcgcaaccatactgggatgagacacgggatgccggtcgcgtcagtgagtccgagatccttgatccggacactggtttcggaggagatggtgtgggtgagaggggttgtatcgccgacggaccgtttgcaggctacatcaacagcatcgggccaggttataggattacggatcgctgcatcacacgcttcgtgaacaacacccgaagcttgatggctagcccccgtttcaccgacaggtgccaaggcatgaatcgatatgttgacgtgtggccatgcctcgagggaaaccctcataattcgggccatggcgctattagcggactgatgatggacccgatcgccagccccggcgaccccatcttctaccttcatcacacatggctcgataagctgtggtgggagtggcaggccatggacctcccccgacgcctcaccgacattggcggccgtaacacccaggacggctccgaaggtttccccggtgcaccgcccaactctaacggtcccaacggcgcgaagcgacgatcccccgccgacggccccatcctcatcttccccggggacggaggaaacattgactgggacgagattgactgggaaaagattggcttccccggcctcggcggtggaaatggaggcccgatccagttgccgcccggcgttgacgttcccccggaagccctggaacctcccgaggatgccgagcctcaagagcccaggggtgacccgggcgatgtgaccacgttgaaccacgtcctgaagatgtttggcctcgttccagacgccttgatccgagacgtcatggatatcgccggaggcactctgtgttacgagtacgtc

SEQ ID NO: 25 - DNA, 990 bp, Paracedosporium putredinis
catacgcatacacttgtgccgcacatgcaagactcaactccctgtatcaacccaagtttgcgacgtccatggcagattctctcggacggcgaaaaacgctcatatctcgatgcccagctttgtgtgatgagaacgccgcagaccctcggtcttcctggcgcgagaacacgtttcgaggagctggctgccacccaccagattggcgcccgtgccagccatgccacggggacatttttcccctaccatcgatacctactgcatgcccatgagtcattgctgaaggagtgcggctaccacgcaggtctcccttactgggatgagaccagggaagctggaaatttcatcaagtctaccatattcgaatcgggcctcggattcggtggctttggaagcgacctcaaagggtgcatcgaagacggacctttcgcaaacttgacaagtacaatcggtcccgggttttcgctgaacgaacactgcatctcacgtgcgctcaatgaaactgcagggctcaaggcggctagggaagaggttgataagtgcttagaagccaacgactatacagagatgtggcgttgtgcatataccacaccccatcgtgggggtcatgggggcgtgggaggcacgatgggagacgctttggcatcgcccggcgacccggtattctacgtccaccacgcttgggtcgataagatttggtgggattggcaggaggctgatcttgataatagaatgtatgctattggcgggcccagcttccagtcacctgatatcgggtttcctgaggttcctggtgatgttgaggaagaagaagcaaatatctttggcaaaccaagcgaagcaatccgacgactacaggagctatggagttcctctgatccaagcagagaaactaccctggagcataatctgacattgctaggtattattcccgacatcaacattagtaaggtcatggacactagaggtgggtatctttgctacgaatatgtt

SEQ ID NO: 26 - protein, 386 aa, Paracedosporium putredinis
QEPALTEDDFSIPEIEGGDALAQLAQLAADSSQETALRMAKRGLNSGCSPSQIKVRREWRTLTSAQRKQYIASVKCLQTKPSFFDPNIIPAAKSLFDDFVGVHVFQTGSIHLTATFLTWHRYFVYTYETKLREECGYTGPLPYWEWGLDVNNPNASPVFDGSDTSLSGNGAFFAHEGIQMVQPINGNILKLPPGNGGGCVTKGPFKDMKVHFGTIILPVYGQPILSGVENPIADNERCLKRDLNAGIAKRFTSFLNSTSVILKNNNIEMFQAHLQGDDRYVLNQLGVHGGGHYTIGGDPGGDPFISPGDPAFYLHHAQIDRIYWIWQMLDFKNRQGVHGTATLQNNPPSANVTVEDTIDLSPLAPPVKIKDLMNTVGGSPLCYIYL

SEQ ID NO: 27 - protein, 364 aa, Paracedosporium putredinis
QTYEFSEEELTSGDALKALSKQAMENALARLPESGEGCTRENVKIRKEWRNMPAEMRIGYVSALQCLMESESEYPDVDGAKTAFDDFAVLHYNLTPFVHNSATFLTFHRYYIHTLEEQMRNKCGYTGDFPYWEWGLDCDDPQQSPLFDGSETSLGSDGEPVEAGAGGGFGGGFGFGMGGGSGGGCVMKGPFSNYTVNLGPSTTADPLAYNPRCIKRNLNGAICKQNASLRNTTTTILDSPDIELFQAIVQGDMRYPEARGLGMAVHGGGHFTIGGDPGGDFYFSPLEPAFFQHHGQIDRMYFVWQNLDWETRQNIAGTGTMMNQPPSPEVEITELLDLSPLAEARPIKDLIDTLGSAPFCFVYE

SEQ ID NO: 28 - protein, 392 aa, Paracedosporium putredinis
SPLNKKATFSYQQVSRNPDFPLDVVDELEAKAMPGVETWMAGKDNANGCTLENAAVRREWGDLSVAEREEYVAAVLCLQKLPSKAPEGKAPGALSRFDDFVATHMTQAMMLHSPTNLFASHRYYIWAYETALREECGYTGYQPYMNYDRYADDLLNSPLFNGNASSLGGNGAPSQYAGVPQPFRPPYNMIPSAGGGGCVTEGPFKDMVVSLGPVGIIVNDIPKNPRSDGLGSNPRCLRRDVNKFSAAGAKANYTYNLITENAGIDAFYNRYLGQPQLKDDPNPWGLHNAGHYIVGGDPGGDFYASPGDPYFYFHHGMLDRVWWIWQMQDPAARVNLIPGTGAPAMNHPGMPMNRRQSSATIVDLGWTAPAVPITELNDSLGGNGGKFCYVYV

SEQ ID NO: 29 - protein, 366 aa, Paracedosporium putredinis
DAVNDLETKGRAALDAVIESSTTCSKDKLKVRREWGDISTTERKAYLDGVLCLLNTPSKLDPARYPGAKNRYDDFVVVHMNQTLSIHGTGNFLVWHRYYVWAWENVMRTECGYEGTQPYWDYGRWAEDPLSSPLFDGSETSLGGNGAPVTQNKRSRVEGRQFGGGGGFGGGLGGGFGGGGDGGGCISTGPFKDMVVTLGPMSAVVRPAPARNPQADGYGSNPRCIRRDITNSLSMAYGKTEDIVNSIVNYNDILAFQNFMQGGTGVHGVGHFTVSGDPGGDFYISPNEPSFWLHHAMIDRIWTIWQSQDYETRRGAMEGGTSMMGGGRAQSLDDLVDLGVIADTVYPIRDILSSVDGPGPFCYVYE

SEQ ID NO: 30 - protein, 377 aa, Paracedosporium putredinis
SSIKKRFSTLDVWRHGDYERDIVDQLSDETFPKIAEWVEKTGSTCTLENAVQRKEWTDLTIDERADYIQAVQCLMKLPPKSQDQVPGSLNRYDDFVATHVTGIPVLHAPTNLFASHRYYIWAYELALREECGYKGYQPYMNYERHQDPITSPLFNGNATSMGGNGAAAEYPGVVMPYPRPYNVIPAAGGGGCVTEGPFSDMVVSIGPLGTVLRDIPRNPRADGLGSNPRCLRRDLNKFSAAGASANHSYSLIMDYPDVDAFYNRYLGQPFLRGDEFPWGLHSAGHYITGGDPGGDFYASPGDPTFWMHHAALDRLWWLWQMQDPENRLQAIPGITSSRMTNEDAQKTMVDLKWTAEPRSLGDLNDQMGSAPFCYIYV

SEQ ID NO: 31 - protein, 336 aa, Paracedosporium putredinis
APTCCTNAERREWRTFSTKEKQAYIAAVKCLQSKPSQLKSTYPTSQNRFDDFQAVHIDLTEKYHFTGPFQAWHRVFLHKYESDLRGLCAYKGYQPYWDWTKDSGSEAAFLASPVFDAVNGFGGNGPYVDTSNFPVTNVPVKIPNKTGGGCVQDGAFVNMTVTLGPGPSLESNPRCLTRDFSYWLISRTLTKAVVDWTLEAASFAVFDFRLQGTGIEPEGMTVHAGGHLGVGGDIGEIGNMYSSPGDPLFYLHHANLDRLWDQWQRKKFAQRVQDMTGPDTMWAYPFNFFGDVPYTNITLETLLDFKGLLGSSSADRYVKIKDVMDSQGPNLCVFYK

SEQ ID NO: 32 - protein, 370 aa, Paracedosporium putredinis
QGPCSTIRQRRAWHTLSNSEKRAYLDAEVCLLGKAPKFGFEGAKNRFEELQAAHQVQAYLIHGVGAFLPFHRYLMHAHETLLRTECGYQGAQPYWDETRDAGRVSESEILDPDTGFGGDGVGERGCIADGPFAGYINSIGPGYRITDRCITRFVNNTRSLMASPRFTDRCQGMNRYVDVWPCLEGNPHNSGHGAISGLMMDPIASPGDPIFYLHHTWLDKLWWEWQAMDLPRRLTDIGGRNTQDGSEGFPGAPPNSNGPNGAKRRSPADGPILIFPGDGGNIDWDEIDWEKIGFPGLGGGNGGPIQLPPGVDVPPEALEPPEDAEPQEPRGDPGDVTTLNHVLKMFGLVPDALIRDVMDIAGGTLCYEYV

SEQ ID NO: 33 - protein, 330 aa, Paracedosporium putredinis
HTHTLVPHMQDSTPCINPSLRRPWQILSDGEKRSYLDAQLCVMRTPQTLGLPGARTRFEELAATHQIGARASHATGTFFPYHRYLLHAHESLLKECGYHAGLPYWDETREAGNFIKSTIFESGLGFGGFGSDLKGCIEDGPFANLTSTIGPGFSLNEHCISRALNETAGLKAAREEVDKCLEANDYTEMWRCAYTTPHRGGHGGVGGTMGDALASPGDPVFYVHHAWVDKIWWDWQEADLDNRMYAIGGPSFQSPDIGFPEVPGDVEEEEANIFGKPSEAIRRLQELWSSSDPSRETTLEHNLTLLGIIPDINISKVMDTRGGYLCYEYV

SEQ ID NO: 34 - DNA, 19 bp, artificial sequence
  primer
tccgtaggtgaacctgcgg

SEQ ID NO: 35 - DNA, 17 bp, artificial sequence
  primer
cgctgcgttcttcatcg

SEQ ID NO: 36 - DNA, 15 bp, artificial sequence
  primer
  y LOCATION: (2)..(2): wherein y is C or T
  y LOCATION: (7)..(7): wherein y is C or T
  d LOCATION: (8)..(8): wherein a is A or G or T
  n LOCATION: (14)..(14): wherein n is A or G or T or C
  misc_feature LOCATION: (14)..(14): n is a, c, g, or t
aytgggydtaaagng

SEQ ID NO: 37 - DNA, 18 bp, artificial sequence
  primer
  n LOCATION: (4)..(4): wherein n is C or T or G or A
  misc_feature LOCATION: (4)..(4): n is a, c, g, or t
  v LOCATION: (5)..(5): wherein v is A or C or G
tacnvgggtatctaatcc

SEQ ID NO: 38 - protein, 391 aa, Agaricus bisporus
SDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYG

SEQ ID NO: 39 - protein, 339 aa, Vitis vinifera
APIQAPDISKCGTATVPDGVTPTNCCPPVTTKIIDFQLPSSGSPMRTRPAAHLVSKEYLAKYKKAIELQKALPDDDPRSFKQQANVHCTYCQGAYDQVGYTDLELQVHASWLFLPFHRYYLYFNERILAKLIDDPTFALPYWAWDNPDGMYMPTIYASSPSSLYDEKRNAKHLPPTVIDLDYDGTEPTIPDDELKTDNLAIMYKQIVSGATTPKLFLGYPYRAGDAIDPGAGTLEHAPHNIVHKWTGLADKPSEDMGNFYTAGRDPIFFGHHANVDRMWNIWKTIGGKNRKDFTDTDWLDATFVFYDENKQLVKVKVSDCVDTSKLRYQYQDIPIPWLP

SEQ ID NO: 40 - protein, 408 aa, Aspergillus oryzea
MVALQALSLGLLASQALAFPAASQQAATATLPTTASSSTAVASSQLDQLANFAYNVTTDSVAGGSESKRGGCTLQNLRVRRDWRAFSKTQKKDYINSVLCLQKLPSRTPAHLAPGARTRYDDFVATHINQTQIIHYTGTFLAWHRYFIYEFEQALRDECSYTGDYPYWNWGADADNMEKSQVFDGSETSMSGNGEYIPNQGDIKLLLGNYPAIDLPPGSGGGCVTSGPFKDYKLNLGPAALSLPGGNMTAAANPLTYNPRCMKRSLTTEILQRYNTFPKIVELILDSDDIWDFQMTMQGVPGSGSIGVHGGGHYSMGGDPGRDVYVSPGDTAFWLHHGMIDRVWWIWQNLDLRKRQNAISGTGTFMNNPASPNTTLDTVIDLGYANGGPIAMRDLMSTTAGPFCYVYL

SEQ ID NO: 41 - protein, 281 aa, Streptomyces castaneoglobisporus
MTVRKNQATLTADEKRRFVAAVLELKRSGRYDEFVRTHNEFIMSDTDSGERTGHRSPSFLPWHRRFLLDFEQALQSVDSSVTLPYWDWSADRTVRASLWAPDFLGGTGRSTDGRVMDGPFAASTGNWPINVRVDSRTYLRRSLGGSVAELPTRAEVESVLAISAYDLPPYNSASEGFRNHLEGWRGVNLHNRVHVWVGGQMATGVSPNDPVFWLHHAYVDKLWAEWQRRHPDSAYVPTGGTPDVVDLNETMKPWNTVRPADLLDHTAYYTFDALEHHHHHH

SEQ ID NO: 42 - protein, 303 aa, Bacillus megaterium
MGNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHDRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSHHHHHH

SEQ ID NO: 43 - protein, 18 aa, artificial sequence
  copper binding site
  X LOCATION: (2)..(8): wherein X is any amino acid
  X LOCATION: (10)..(17): wherein X is any amino acid
HXXXXXXXHXXXXXXXXH

SEQ ID NO: 44 - protein, 28 aa, artificial sequence
  copper binding site
  X LOCATION: (2)..(4): wherein X is any amino acid
  X LOCATION: (6)..(27): wherein X is any amino acid
HXXXHXXXXXXXXXXXXXXXXXXXXXXH

SEQ ID NO: 45 - protein, 29 aa, artificial sequence
  copper binding site
  X LOCATION: (2)..(4): wherein X is any amino acid
  X LOCATION: (6)..(28): wherein X is any amino acid
HXXXHXXXXXXXXXXXXXXXXXXXXXXXH

SEQ ID NO: 46 - protein, 30 aa, artificial sequence
  copper binding site
  X LOCATION: (2)..(4): wherein X is any amino acid
  X LOCATION: (6)..(29): wherein X is any amino acid
HXXXHXXXXXXXXXXXXXXXXXXXXXXXXH

SEQ ID NO: 47 - protein, 31 aa, artificial sequence
  copper binding site
  X LOCATION: (2)..(4): wherein X is any amino acid
  X LOCATION: (6)..(30): wherein X is any amino acid
HXXXHXXXXXXXXXXXXXXXXXXXXXXXXXH

(19) United States
(12) Patent Application Publication
(10) Pub. No.: US 2023/0203461 A1
(43) Pub. Date: Jun. 29, 2023

(54) BETA-ETHERASES FOR LIGNIN DEPOLYMERISATION

(71) Applicants: The University of York, York (GB); Wisconsin Alumni Research Foundation, Madison, WI (US)

(72) Inventors: Neil Bruce, York (GB); Nicola Oates, York (GB); John Ralph, Madison, WI (US)

(73) Assignees: The University of York, York (GB); Wisconsin Alumni Research Foundation, Madison, WI (US)

(21) Appl. No.: 17/791,144

(22) PCT Filed: Jan. 9, 2021
(86) PCT No.: PCT/EP2021/050317

§ 371 Date: Jul. 6, 2022

(30) Foreign Application Priority Data
Jan. 10, 2020 (GB) 2000378.6

(51) Int. Cl. C12N 9/14; C12N 15/80

(52) U.S. Cl. CPC C12N 9/14; C12N 15/80; C12Y 303/00

(57) ABSTRACT

The present application relates to nucleic acids encoding polypeptides with β-etherase activity; polypeptides with β-etherase activity; vectors comprising said nucleic acids for the production of recombinant β-etherase; cells, for example microbial cells transformed with nucleic acids encoding β-etherase activity and vectors, including nucleic acids encoding β-etherases; a composition comprising β-etherases suitable for processing lignocellulose and a method that uses β-etherases or compositions comprising β-etherases in the processing of lignocellulose and related polysaccharides.

### FIELD OF THE DISCLOSURE

The present application relates to nucleic acids encoding polypeptides with β-etherase activity; polypeptides with β-etherase activity; vectors comprising said nucleic acids for the production of recombinant β-etherase; cells, for example microbial cells, transformed with nucleic acids encoding β-etherase activity and vectors including nucleic acids encoding β-etherases; a composition comprising β-etherases suitable for processing lignocellulose; and a method that uses β-etherases or compositions comprising β-etherases in the processing of lignocellulose and related polysaccharides.

### GOVERNMENT RIGHTS

This invention was made with government support under DE-SC0018409 awarded by the US Department of Energy. The government has certain rights in the invention.

### BACKGROUND TO THE DISCLOSURE

The plant cell wall is composed of cellulose, hemicelluloses, pectic polysaccharides, and lignin, and is collectively termed lignocellulose. Photosynthetically fixed carbon in lignocellulose is produced in vast quantities on the Earth’s surface. Its conversion into liquid transportation fuel represents a potential source of renewable energy with diverse feedstocks, including agricultural residues, municipal waste, and dedicated low-input crops. Effective utilization of lignocellulose, nevertheless, remains a challenge, as the extraction of fermentable sugars for biofuel production requires intensive physico-chemical pretreatments and high loadings of enzyme cocktails. A key factor of this recalcitrance to degradation is the presence of lignin, a heterogeneous, hydrophobic aromatic polymer that encases the cellulose and hemicellulose, blocking enzyme accessibility and impeding cellulase activity.

Lignin is synthesised by plants through the oxidative coupling of three hydroxycinnamyl alcohols: coniferyl alcohol, sinapyl alcohol and p-coumaryl alcohol, generating β—O—4, 4—O—5, β-5, β-1, 5-5 and β-β inter-unit linkages in β-ether, biphenyl ether, phenylcoumaran, spirodienone, biphenyl, and resinol units, respectively. Lignin requires a high redox potential to be oxidatively attacked. Recalcitrance to degradation is further enhanced as lignin has no defined repeat structure. The β—O—4 (or β-aryl) ether linkage is the most abundant linkage in the lignin macromolecule; its cleavage results in substantial lignin depolymerization.

Enzymes for depolymerising lignin are known and disclosed in US2019/048329 and include dehydrogenases, glutathione lyases and β-etherases which attack β—O—4 ether linkages. The β-etherase activity disclosed in US2019/048329 requires the co-substrates NAD⁺ and glutathione.

Tricin, [5,7-dihydroxy-2-(4-hydroxy-3,5-dimethoxyphenyl)-4H-chromen-4-one], an O-methylated flavone, forms part of the structure of lignin from monocot plants including wheat, rice, sugar cane, and palms. Tricin has only been observed incorporated into the lignin structure via 4—O—β linkages, having arisen from the radical coupling of the flavone at its 4′—O—position with the monolignol at its β-position.

Tricin is recognized as a valuable human health compound due to its antioxidant, anti-aging, anti-cancer, and cardio-protective potential. Tricin may be present as its parent compound that may be released by solvent extraction from a variety of monocotyledons such as wheat (Triticum aestivum), oat bran (Avena sativa), bamboo (Leleba oldhami), sugarcane (Saccharum officinarum), and maize (Zea mays), and has been observed in quantities of up to 3.3% wt of lignin from wheat straw.

This disclosure characterises a copper-containing β-etherase that can cleave the β-aryl ether linkage of lignin and which is secreted from the fungus Parascedosporium when growing on wheat straw. The disclosed β-etherase has no requirement for NAD⁺ and/or glutathione and was found to readily cleave tricin from wheat straw, also enhancing the saccharification of lignocellulosic biomass when used in combination with cellulolytic enzymes.

### STATEMENTS OF THE INVENTION

According to an aspect of the invention there is provided an isolated nucleic acid molecule encoding a β-etherase polypeptide wherein said polypeptide comprises copper and further wherein the activity of said polypeptide is independent of NAD⁺ and/or glutathione.

Lignin, the major component of lignocellulosic plant biomass, is an organic heterologous polymer comprising covalently linked phenylpropanoid units and consist essentially of crosslinked methoxylated derivatives of benzene such as p-coumaryl, coniferyl, and sinapyl alcohols. Exemplary phenylpropanoid units derived from the alcohols are p-hydroxyphenyl, guaiacyl, and syringyl units respectively. The phenylpropanoid units can be linked to other phenylpropanoid units through bonds such as β—O—4, 4—O—5, β-5, β-1, 5-5 and β-β inter-unit linkages. β—O—4 ether bonds account for 45-60% of linkages present in lignin. Flavonoid units such as tricin can be incorporated into lignin via 4—O—β ether bonds.

β-etherase activity in the context of this application refers to the capability to cleave β-aryl ether (β—O—4) bonds in lignin that link one phenylpropanoid unit to another phenylpropanoid unit or to flavonoid units such as tricin.

In order to optimize expression levels in recombinant host cells, codon optimisation of the nucleic acid sequence to be expressed may be required to convert a natural sequence to a non-natural sequence that encodes substantially the same polypeptide and would be optimally expressed in a heterologous host cell. Codon optimisation is known in the art and increases translational efficiency in the desired host organism and replace codons of low frequency with codons of high frequency.

In a preferred embodiment of the invention, the said isolated nucleic acid molecule comprises a nucleotide sequence selected from the group consisting of:
i) a nucleotide sequence as set forth in SEQ ID NO: 1;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to sequence set forth in SEQ ID NO 1;
iv) a nucleotide sequence that encodes a polypeptide comprising an amino acid sequence set forth in SEQ ID NO 9;
v) a nucleotide sequence that encodes a polypeptide comprising an amino acid sequence wherein said amino acid sequence is modified by addition, deletion or substitution of at least one amino acid residue as represented in iv) above and has β-etherase activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology-Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:
Very High Stringency (allows sequences that share at least 90% or 95% identity to hybridize)
   Hybridization: 5x SSC at 65° C. for 16 hours
   Wash twice: 2x SSC at room temperature (RT) for 15 minutes each
   Wash twice: 0.5x SSC at 65° C. for 20 minutes each
High Stringency (allows sequences that share at least 80% identity to hybridize)
   Hybridization: 5x-6x SSC at 65-70° C. for 16-20 hours
   Wash twice: 2x SSC at RT for 5-20 minutes each
   Wash twice: 1x SSC at 55-70° C. for 30 minutes each
Low Stringency (allows sequences that share at least 50% identity to hybridize)
   Hybridization: 6x SSC at RT to 55° C. for 16-20 hours
   Wash at least twice: 2x-3x SSC at RT to 55° C. for 20-30 minutes each.

In a preferred embodiment of the invention said isolated nucleic acid molecule comprises a nucleotide sequence selected from the group consisting of:
i) a nucleotide sequence as set forth in SEQ ID NO: 2;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 2;
iv) a nucleotide sequence that encodes a polypeptide comprising an amino acid sequence set forth in SEQ ID NO: 10;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence set forth in SEQ ID NO: 3;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 3;
iv) a nucleotide sequence that encodes a polypeptide comprising an amino acid sequence as set forth in SEQ ID NO 11;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 4;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 4;
iv) a nucleotide sequence that encodes a polypeptide comprising an amino acid sequence as set forth in SEQ ID NO 12:
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 5:
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 5;
iv) a nucleotide sequence that encodes a polypeptide comprising an amino acid sequence as represented in SEQ ID NO 13;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleic acid sequences as set forth in SEQ ID NO 6;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 6;
iv) a nucleotide sequence that encodes a polypeptide comprising an amino acid sequence as set forth in SEQ ID NO 14;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprising a nucleotide sequence selected from the group consisting of:
i) a nucleotide sequence as set forth in SEQ ID NO: 7;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions set forth in SEQ ID NO 7;
iv) a nucleotide sequence that encodes a polypeptide comprising an amino acid sequence as set forth SEQ ID NO 15;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 8;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 8;
iv) a nucleotide sequence that encodes a polypeptide comprising an amino acid sequence set forth in SEQ ID NO 16;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 18 or 17;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 18 or 17;
iv) a nucleotide sequence that encodes a polypeptide comprising the amino acid sequence set forth in SEQ ID NO 26;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 19;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 19;
iv) a nucleotide sequence that encodes a polypeptide comprising an amino acid sequence set forth in SEQ ID NO 27;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 20;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 20;
iv) a nucleotide sequence that encodes a polypeptide comprising an amino acid sequence set forth in SEQ ID NO 28;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 21;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 21;
iv) a nucleotide sequence that encodes a polypeptide comprising an amino acid sequence set forth in SEQ ID NO 29;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 22;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 22;
iv) a nucleotide sequence that encodes a polypeptide comprising an amino acid sequence set forth in SEQ ID NO 30;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 23;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 23;
iv) a nucleotide sequence that encodes a polypeptide comprising an amino acid sequence set forth in SEQ ID NO 31;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 24;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 24;
iv) a nucleotide sequence that encodes a polypeptide comprising an amino acid sequence set forth in SEQ ID NO 32;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 24;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 24;
iv) a nucleotide sequence that encodes a polypeptide comprising an amino acid sequence set forth in SEQ ID NO 32;
v) a nucleotide sequence that encodes a polypeptide comprising an amino acid sequence wherein said amino acid sequence is modified by addition, deletion or substitution of at least one amino acid residue as represented in iv) above and has β-etherase activity.

In a preferred embodiment of the invention said isolated nucleic acid molecule comprises a nucleotide sequence selected from the group consisting of:
i) a nucleotide sequence as set forth in SEQ ID NO 25;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to the sequence set forth in SEQ ID NO 25;
iv) a nucleotide sequence that encodes a polypeptide comprising an amino acid sequence set forth in SEQ ID NO 33;
v) a nucleotide sequence that encodes a polypeptide comprising an amino acid sequence wherein said amino acid sequence is modified by addition, deletion or substitution of at least one amino acid residue as represented in iv) above and has β-etherase activity.

The presence of a peptide signal sequence encoded by part of the nucleic acid sequence set forth in SEQ ID NO 1-8 which is located at the N-terminus of the amino acid sequences set forth in SEQ ID NO 9-16, may result in inefficient expression of the protein in an alternative expression host cell. Therefore, typically, the endogenous host specific signal sequence is either replaced with the expression host specific peptide signal sequence or with an ATG codon. The nucleotide sequences set forth in sequence IDs 17-25 represent the nucleotide sequence lacking the signal sequence or an ATG start codon at the 5′-end of the nucleotide sequence and correspondingly, the amino acid sequences set forth in SEQ IDs No 26-33 are lacking the N-terminal signal sequence or a methionine as the first amino acid at the N-terminus of the amino acid sequence. Thus, nucleotide sequences set forth in SEQ ID NO 17-25 comprising an ATG as the first codon at the 5′-end or amino acid sequences set forth in SEQ ID NO 26-33 comprising a methionine as the first amino acid of the N-terminus are also claimed.

In a preferred embodiment of the invention said nucleic acid molecule comprises or consists of a nucleotide sequence as set forth in SEQ ID NO: 1 wherein said nucleic acid molecule encodes a polypeptide with β-etherase activity.

In a preferred embodiment of the invention said nucleic acid molecule comprises or consists of a nucleotide sequence as set forth in SEQ ID NO: 2 wherein said nucleic acid molecule encodes a polypeptide with β-etherase activity.

In a preferred embodiment of the invention said nucleic acid molecule comprises or consists of a nucleotide sequence as set forth in SEQ ID NO: 3 wherein said nucleic acid molecule encodes a polypeptide with β-etherase activity.

In a preferred embodiment of the invention said nucleic acid molecule comprises or consists of a nucleotide sequence as set forth in SEQ ID NO: 4 wherein said nucleic acid molecule encodes a polypeptide with β-etherase activity

In a preferred embodiment of the invention said nucleic acid molecule comprises or consists of a nucleotide sequence as set forth in SEQ ID NO: 5 wherein said nucleic acid molecule encodes a polypeptide with β-etherase activity.

In a preferred embodiment of the invention said nucleic acid molecule comprises or consists of a nucleotide sequence as set forth in SEQ ID NO: 6 wherein said nucleic acid molecule encodes a polypeptide with β-etherase activity.

In a preferred embodiment of the invention said nucleic acid molecule comprises or consists of a nucleotide sequence as set forth in SEQ ID NO: 7 wherein said nucleic acid molecule encodes a polypeptide with β-etherase activity.

In a preferred embodiment of the invention said nucleic acid molecule comprises or consists of a nucleotide sequence as set forth in SEQ ID NO: 8 wherein said nucleic acid molecule encodes a polypeptide with β-etherase activity.

In a preferred embodiment of the invention said nucleic acid molecule comprises or consists of a nucleotide sequence as set forth in SEQ ID NO: 17 wherein said nucleic acid molecule encodes a polypeptide with β-etherase activity.

In a preferred embodiment of the invention said nucleic acid molecule comprises or consists of a nucleotide sequence as set forth in SEQ ID NO: 18 wherein said nucleic acid molecule encodes a polypeptide with β-etherase activity.

In a preferred embodiment of the invention said nucleic acid molecule comprises or consists of a nucleotide sequence as set forth in SEQ ID NO: 19 wherein said nucleic acid molecule encodes a polypeptide with β-etherase activity.

In a preferred embodiment of the invention said nucleic acid molecule comprises or consists of a nucleotide sequence as set forth in SEQ ID NO: 20 wherein said nucleic acid molecule encodes a polypeptide with β-etherase activity.

In a preferred embodiment of the invention said nucleic acid molecule comprises or consists of a nucleotide sequence as set forth in SEQ ID NO: 21 wherein said nucleic acid molecule encodes a polypeptide with β-etherase activity.

In a preferred embodiment of the invention said nucleic acid molecule comprises or consists of a nucleotide sequence as set forth in SEQ ID NO: 22 wherein said nucleic acid molecule encodes a polypeptide with β-etherase activity.

In a preferred embodiment of the invention said nucleic acid molecule comprises or consists of a nucleotide sequence as set forth in SEQ ID NO: 23 wherein said nucleic acid molecule encodes a polypeptide with β-etherase activity.

In a preferred embodiment of the invention said nucleic acid molecule comprises or consists of a nucleotide sequence as set forth in SEQ ID NO: 24 wherein said nucleic acid molecule encodes a polypeptide with β-etherase activity.

In a preferred embodiment of the invention said nucleic acid molecule comprises or consists of a nucleotide sequence as set forth in SEQ ID NO: 25 wherein said nucleic acid molecule encodes a polypeptide with β-etherase activity.

According to a further aspect of the invention there is provided an isolated β-etherase polypeptide wherein said polypeptide comprises copper and further wherein the activity of said polypeptide is independent of NAD⁺ and/or glutathione.

In a preferred embodiment of the invention said β-etherase polypeptide comprises two copper binding sites comprising the motif:
Copper binding site No 1: H—X(1-7)—H—X(1-8)—H and site No 2:H— X(1-3)—H—X(22-25)—H;
wherein X is any amino acid and H is histidine. The numerical range X (1-7), X (1-8), X (1-3) and X (22-25) denotes the number of amino acid residues between the histidines e.g., H—X (1-3)—H contains three amino acid residues between the two histidines. Variations to this motif are shown in FIG. 11.

In a preferred embodiment of the invention said polypeptide has β-etherase activity in the absence of NAD⁺ and glutathione.

In a further preferred embodiment of the invention said isolated β-etherase polypeptides share at least 23% sequence identity over the full-length sequence set forth in SEQ ID NO 9 or 26

In a further preferred embodiment of the invention said isolated β-etherase polypeptides share between 23-45% sequence identity over the full-length sequence set forth in SEQ ID NO 9 or 26.

In a further preferred embodiment of the invention said isolated β-etherase polypeptides share at least 23%, 24%, 25%, 30%, 35%, 37%, 38%, 39%, 40%, 41%, 44% and 45% sequence identity over the full-length sequence set forth in SEQ ID NO 9 or 26.

In an alternative further preferred embodiment of the invention said isolated β-etherase polypeptides share at least 50% sequence identity over the full-length sequence set forth in SEQ ID NO 9 or 26.

In an alternative further preferred embodiment of the invention said isolated β-etherase polypeptides share between 50-88% sequence identity over the full-length sequence set forth in SEQ ID NO 9 or 26.

In an alternative further preferred embodiment of the invention said isolated β-etherase polypeptides share at least 50%, 55%, 60%, 65%, 70%, 75%, 80%, 85%, 86%, 87%, 88%, 89%, 90%, 91%, 92%, 93%, 94%, 95%, 96%, 97%, 98% and 99% sequence identity over the full-length sequence set forth in SEQ ID NO 9 or 26.

In a preferred embodiment of the invention said isolated polypeptide is selected from the group consisting of:
i) a polypeptide comprising or consisting of an amino acid sequence as set forth in SEQ ID NO: 9 or 26;
ii) a modified polypeptide comprising or consisting of a modified amino acid sequence wherein said polypeptide is modified by addition, deletion or substitution of at least one amino acid residue of the sequence set forth in SEQ ID NO: 9 or 26 and which has β-etherase activity.

In a preferred embodiment of the invention said isolated polypeptide is selected from the group consisting of:
i) a polypeptide comprising or consisting of an amino acid sequence as set forth in SEQ ID NO: 10 or 27;
ii) a modified polypeptide comprising or consisting of a modified amino acid sequence wherein said polypeptide is modified by addition, deletion or substitution of at least one amino acid residue of the sequence set forth in SEQ ID NO: 10 or 27 and which has β-etherase activity.

According to an aspect of the invention there is provided an isolated polypeptide selected from the group consisting of:
i) a polypeptide comprising or consisting of an amino acid sequence as set forth in SEQ ID NO: 11 or 28;
ii) a modified polypeptide comprising or consisting of a modified amino acid sequence wherein said polypeptide is modified by addition, deletion or substitution of at least one amino acid residue of the sequence set forth in SEQ ID NO: 11 or 28 and which has β-etherase activity.

In a preferred embodiment of the invention said isolated polypeptide is selected from the group consisting of:
i) a polypeptide comprising or consisting of an amino acid sequence as set forth in SEQ ID NO: 12 or 29;
ii) a modified polypeptide comprising or consisting of a modified amino acid sequence wherein said polypeptide is modified by addition, deletion or substitution of at least one amino acid residue of the sequence set forth in SEQ ID NO: 12 or 29 and which has β-etherase activity.

In a preferred embodiment of the invention said isolated polypeptide is selected from the group consisting of:
i) a polypeptide comprising or consisting of an amino acid sequence as set forth in SEQ ID NO: 13 or 30;
ii) a modified polypeptide comprising or consisting of a modified amino acid sequence wherein said polypeptide is modified by addition, deletion or substitution of at least one amino acid residue of the sequence set forth in SEQ ID NO: 13 or 30 and which has β-etherase activity.

In a preferred embodiment of the invention said isolated polypeptide is selected from the group consisting of:
i) a polypeptide comprising or consisting of an amino acid sequence as set forth in SEQ ID NO: 14 or 31;
ii) a modified polypeptide comprising or consisting of a modified amino acid sequence wherein said polypeptide is modified by addition, deletion or substitution of at least one amino acid residue of the sequence set forth in SEQ ID NO: 14 or 31 and which has β-etherase activity.

In a preferred embodiment of the invention said isolated polypeptide is selected from the group consisting of:
i) a polypeptide comprising or consisting of an amino acid sequence as set forth in SEQ ID NO: 15 or 32;
ii) a modified polypeptide comprising or consisting of a modified amino acid sequence wherein said polypeptide is modified by addition, deletion or substitution of at least one amino acid residue of the sequence set forth in SEQ ID NO: 15 or 32 and which has β-etherase activity.

In a preferred embodiment of the invention said isolated polypeptide is selected from the group consisting of:
i) a polypeptide comprising or consisting of an amino acid sequence set forth in SEQ ID NO: 16 or 33
ii) a modified polypeptide comprising or consisting of a modified amino acid sequence wherein said polypeptide is modified by addition, deletion or substitution of at least one amino acid residue of the sequence set forth in SEQ ID NO: 16 or 33 and which has β-etherase activity.

A modified polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain the same biological function and activity as the reference polypeptide from which it varies.

In a preferred embodiment of the invention the modified polypeptides have at least 23%, 24%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% identity, and at least 99% identity with the full-length amino acid sequence illustrated herein.

In a preferred embodiment of the invention the modified polypeptides have at least 23% identity with the full-length amino acid sequence illustrated herein.

In a preferred embodiment of the invention the modified polypeptides have at least 88% identity with the full-length amino acid sequence illustrated herein.

According to a further aspect of the invention there is provided a vector comprising a nucleic acid molecule according to the invention.

In a preferred embodiment of the invention the vector is an expression vector adapted for expression in a microbial host cell as herein disclosed.

Preferably the nucleic acid molecule in the vector is under the control of, and operably linked to, an appropriate promoter or other regulatory elements for transcription in a host cell such as a microbial, (e.g., bacterial, yeast), or plant cell. The vector may be a bi-functional expression vector which functions in multiple hosts.

According to a further aspect of the invention there is provided a host cell transformed or transfected with a nucleic acid molecule or vector according to the invention. In a preferred embodiment of the invention said cell is a heterologous host cell wherein said heterologous host cell does not naturally express a nucleic acid molecule according to the invention or vector comprising a nucleic acid molecule according to the invention.

In a further preferred embodiment of the invention said cell transformed or transfected with a nucleic acid molecule or vector according to the invention is a recombinant cell.

In the context of this application a recombinant cell defines a host organism cell comprising DNA from a different species e.g. expression of a nucleotide sequence from Parascedosporium species in an Aspergillus spp cell. In a preferred embodiment of the invention said cell is a microbial cell.

In a preferred embodiment said cell is selected from the group consisting of bacterial cell, yeast cell, fungal cell, insect cell and plant cell.

In a preferred embodiment said cell is a bacterial cell.

In a preferred embodiment of the invention said bacterial cell is an Escherichia coli cell.

In a preferred embodiment said transgenic is a fungal or yeast cell.

In a further preferred embodiment of the invention said fungal cell is an Aspergillus sp. cell

In a further preferred embodiment of the invention said fungal cell is an Aspergillus niger cell.

In a further preferred embodiment of the invention said fungal cell is not a Parascedosporium sp cell.

In a preferred embodiment of the invention said yeast cell is selected from the group consisting of Saccharomyces cerevisae, Schizosaccharomyces pombe or Pichia pastoris.

If microbial cells are used as organisms and in the process according to the invention they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulphate, trace elements such as salts of iron, copper, manganese and magnesium and, if appropriate, vitamins, at temperatures of between 0° C. and 100° C., preferably between 10° C. and 60° C., while gassing in oxygen.

The pH of the liquid medium can either be kept constant and regulated during the culturing period, or not. The cultures can be grown batchwise, semi-batchwise or continuously. Nutrients can be provided at the beginning of the fermentation or fed in semi-continuously or continuously. To this end, the organisms can advantageously be disrupted beforehand. In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulphate, ammonium chloride, ammonium phosphate, ammonium carbonate or ammonium nitrate, nitrates, urea, amino acids, or complex nitrogen sources such as cornsteep liquor, soya meal, soya protein, yeast extract, meat extract, and others. The nitrogen sources can be used individually or as a mixture.

Inorganic salt compounds which may be present in the media comprise the chloride, phosphorus and sulphate salts of calcium, magnesium, sodium, cobalt, molybdenum, potassium, manganese, zinc, copper, and iron.

Inorganic sulphur-containing compounds such as, for example, sulphates, sulphites, dithionites, tetrathionates, thiosulfates, sulphides, or else organic sulphur compounds such as mercaptans and thiols may be used as sources of sulphur for the production of sulphur-containing fine chemicals and pathway intermediates, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate, and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P.M. Rhodes, P.F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C. and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

According to an aspect of the invention there is provided a method for the manufacture of a β-etherase polypeptide comprising the following steps:
i) provide a cell according to the invention and cell culture medium,
ii) culture the host cell in i) above to express the polypeptide according to the invention; and optionally,
iii) isolating said polypeptide from the cell or cell culture medium.

In a preferred method of the invention said cell is a microbial cell.

Preferably, said microbial cell is a bacterial or fungal host cell.

Protocols for the manufacture of recombinantly expressed proteins are known to the skilled person. Isolating proteins under denaturing conditions can result in a higher yield of the protein of interest when compared to non-denaturing protein purification methods. The purified denatured proteins are subsequently allowed to re-fold into their native structure.

In a further method said polypeptide isolation is under denaturing conditions.

According to an aspect of the invention there is provided a composition comprising or consisting of one or more polypeptides according to the invention.

In a preferred embodiment of the invention said composition comprises at least the polypeptide is set forth in SEQ ID NO:9 or 26

In a further preferred embodiment of the invention said one more polypeptide is set forth in SEQ ID NO: 9, 10, 11, 12, 13, 14, 15 and 16.

In a further preferred embodiment of the invention said one more polypeptide is set forth in SEQ ID NO: 26, 27, 28, 29, 30, 31, 32 and 33.

In a further preferred embodiment of the invention said composition further comprises one or more polypeptides for the saccharification of lignocellulose selected from the group consisting of cellulases, lytic polysaccharide monooxygenases, carbohydrate esterases, hemicellulases, glycosylhydrolases, endoglucanases, cellobiohydrolases, beta-glucosidases, xylanases, mannases, cellobiose dehydrogenases, and beta-xylosidases.

Saccharification is the process of breaking down complex carbohydrates such as cellulose into polysaccharides, disaccharides, and monosaccharides.

In a further preferred embodiment of the invention said composition comprises a buffer.

In a preferred embodiment of the invention said composition has a pH between 5 and 12, more preferably between 6 and 11, even more preferably between 7 and 10.

In a preferred embodiment of the invention said composition has a pH of 10.

In a preferred embodiment of the invention said composition has a pH of 7.

According to an aspect of the invention there is provided a method for the modification of plant biomass comprising the following steps:
I) contacting plant biomass with a composition or cell according to the invention to form a reaction mixture and
II) incubating said reaction mixture under conditions which cleaves β-ether linkages present the plant biomass to obtain depolymerised lignin units.

Plant biomass in the context of this application comprises or consist of lignin and/or lignocellulose.

In a preferred method of the invention said method comprises further step iii) extracting said depolymerised lignin units from the reaction mixture.

In a preferred method of the invention said depolymerised lignin units are selected from the group consisting of flavones, p-coumaric acid, and ferulic acid.

In a further preferred method of the invention said depolymerised lignin units are selected from the group consisting of flavones and p-coumaric acid.

In a further preferred method of the invention said depolymerised lignin units are selected from the group consisting of flavones, monomeric guaiacyl phenylpropanoid units, monomeric syringyl phenylpropanoid units, and monomeric p-hydroxyphenyl phenylpropanoid units.

In a further preferred method of the invention said flavones are tricin.

In a further preferred method of the invention said depolymerised lignin units are tricin and/or p-coumaric acid.

In a further preferred method of the invention said plant biomass is selected from hardwood and softwood or woody biomass.

In the context of this application woody biomass defines saw mill or paper mill discards.

In a further preferred method of the invention said plant biomass is selected from grasses, corn stover, corncob, corn fiber, wheat straw, sugarcane bagasse, wood pulp, rice straw, and municipal solid waste.

In a further preferred method of the invention said plant biomass is wheat straw or sugarcane bagasse.

In a further preferred method of the invention said method comprises further step of contacting the reaction mixture of iii) with a saccharification composition comprising one or more polypeptides for the saccharification of depolymerised lignin units.

In a preferred further method of the invention said saccharification composition comprises or consist of one or more polypeptides selected from the group consisting of cellulases, lytic polysaccharide monooxygenases, carbohydrate esterases, hemicellulases, glycosylhydrolases, endoglucanases, cellobiohydrolases, beta-glucosidases, xylanases, mannases, cellobiose dehydrogenases, and beta-xylosidases

In an alternative preferred method of the invention said saccharification composition is provided during step i).

In a preferred method of the invention said method comprises extracting di- and/or monosaccharides.

In a preferred method of the invention said monosaccharides are selected from the group consisting of glucose, xylose, and arabinose

According to an aspect of the invention there is provided the use of the polypeptides, cells or composition according to the invention in the hydrolysis of lignocellulose.

According to a further aspect of the invention there is provided a bioreactor comprising a cell or composition according to the invention.

In a preferred embodiment of the invention said bioreactor is a fermenter.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures:

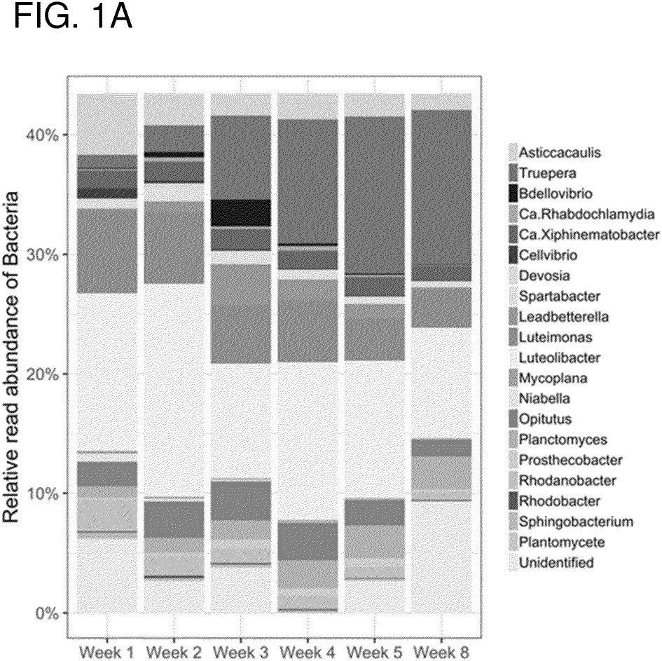

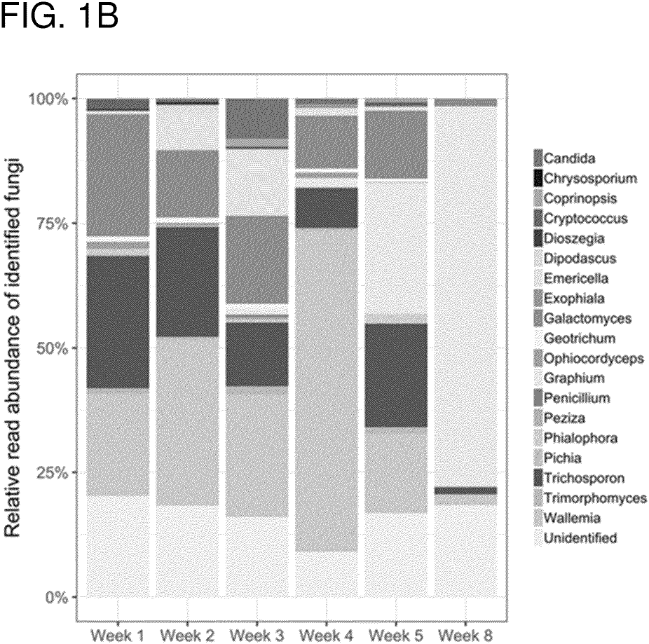

FIG. 1. Composition of prokaryotic and eukaryotic genera during wheat straw degradation. Sequences were generated on an ion torrent platform after amplification of the 16S and ITS for a) prokaryotic and b) eukaryotic identification, respectively. Operational taxonomic units were identified to genus level N=1;

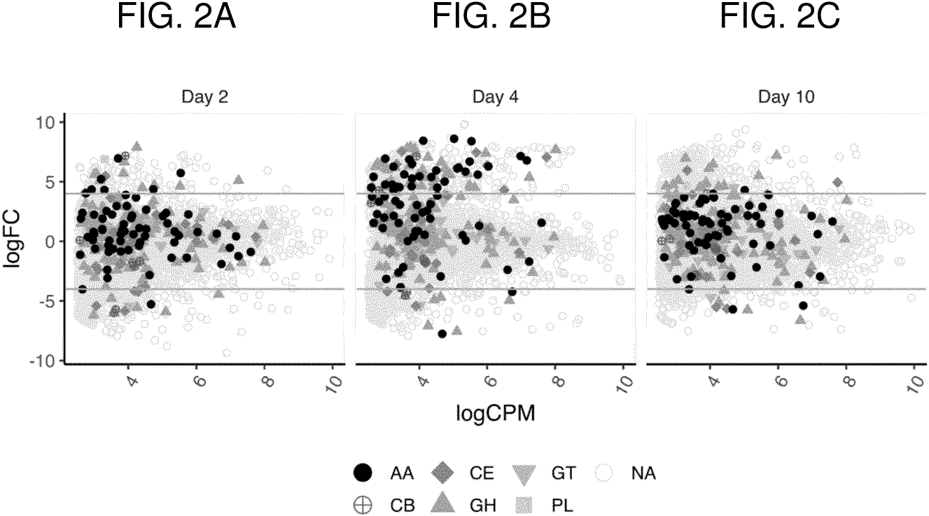

FIG. 2. Expression change of contigs between glucose and wheat straw conditions. RNA was extracted and sequenced after a) two, b) four and c) ten days of P. putredinis NO1. incubation on wheat straw and four days of growth on glucose. Points represent the log fold change (FC) and average counts per million (CPM) of contigs, between the wheat straw and glucose conditions. Carbohydrate-active enzymes were annotated using dbCAN namely auxiliary activities (AA), glycoside hydrolases (GH), polysaccharide lyases (PL), carbohydrate esterases (CE), glycosyltransferases (GT), and non-catalytic carbohydrate-binding modules (CB). Points are the average of three biological replicates;

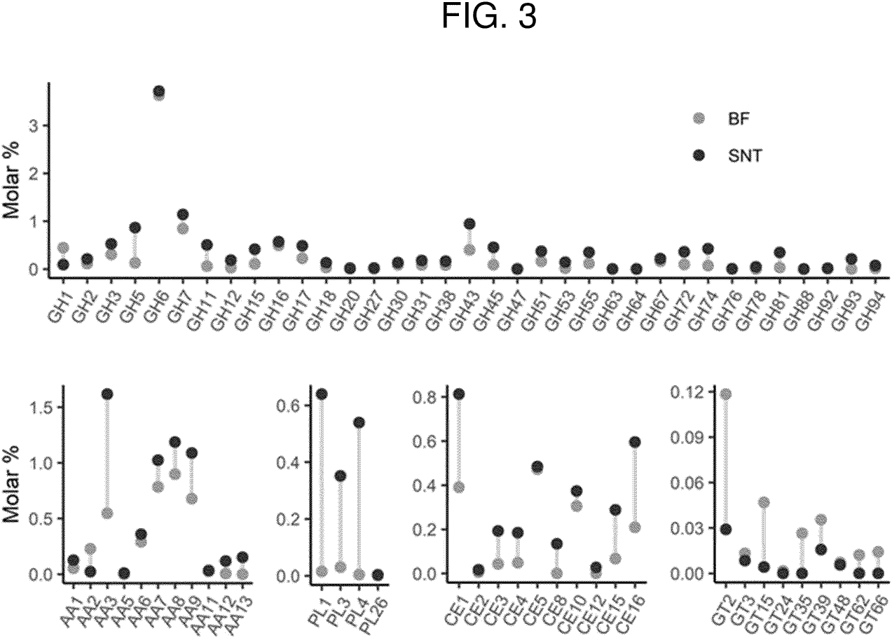

FIG. 3. Molar percentages of supernatant (SNT) and biotin-labelled (BF) proteins after four days of incubation on wheat straw. Molar percentages of carbohydrate-active families, GH: Glycoside hydrolase, AA: Auxiliary activity, PL: Polysaccharide lyase, CE: Carbohydrate esterase, and GT glycosyl transferase, were calculated as the sum of contigs annotated and taken as an average for each biological replicate. N=3;

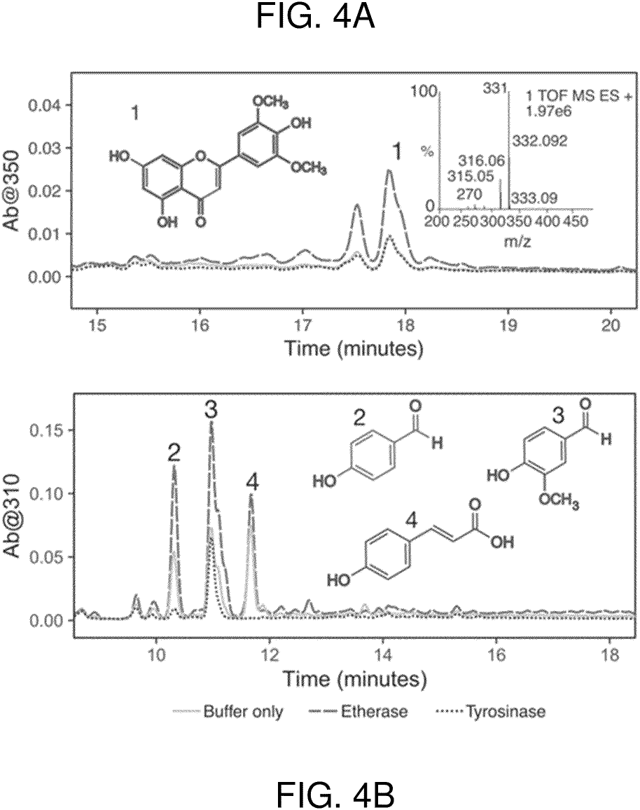

FIG. 4. Release of compounds after incubation with lignocellulosic biomasses. Biomass was treated for 16 h with our recombinant β-etherase, mushroom tyrosinase, and buffer alone, and reaction products were extracted with ethyl acetate, a) Tricin 1 release from wheat straw was observed and compared to an authentic standard using a High-Performance Liquid-Chromatography (HPLC), and mass was confirmed by time-of-flight mass spectrometry. b) HPLC analysis of enzyme incubations with sugarcane bagasse. Products were identified by mass spectrometry and comparison with authentic standards, as p-hydroxybenzaldehyde 2, vanillin 3, p-coumaric acid 4;

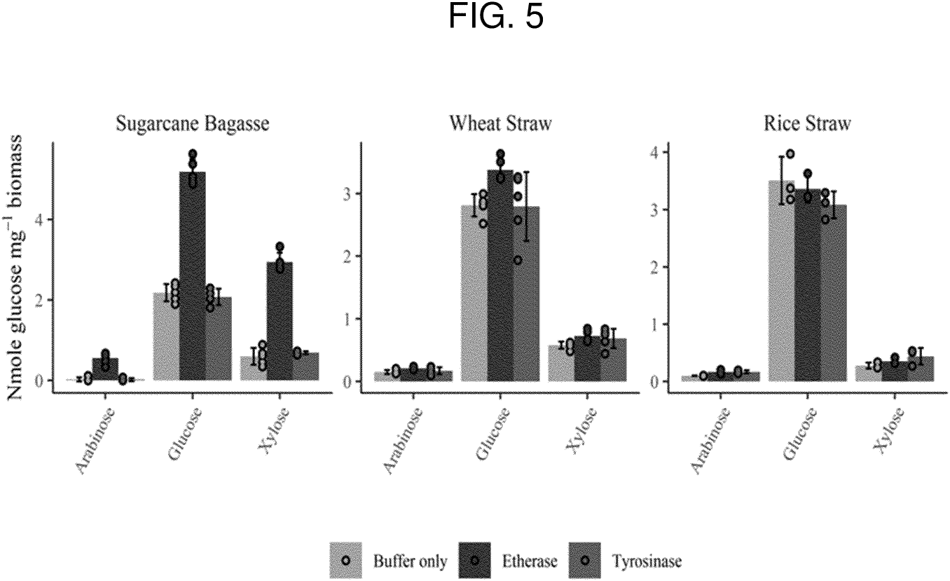

FIG. 5. Release of sugars from sugarcane bagasse, wheat straw, and rice straw. Sugarcane bagasse, wheat straw, and rice straw were treated with recombinant β-etherase, commercial mushroom tyrosinase, and buffer only for 16 h prior to the application of Celluclast® commercial saccharification cocktail. Sugar release was calculated from the reaction mixture using High-Performance Anion-Exchange chromatography. Error bars represent the standard deviation of five biological replicates;

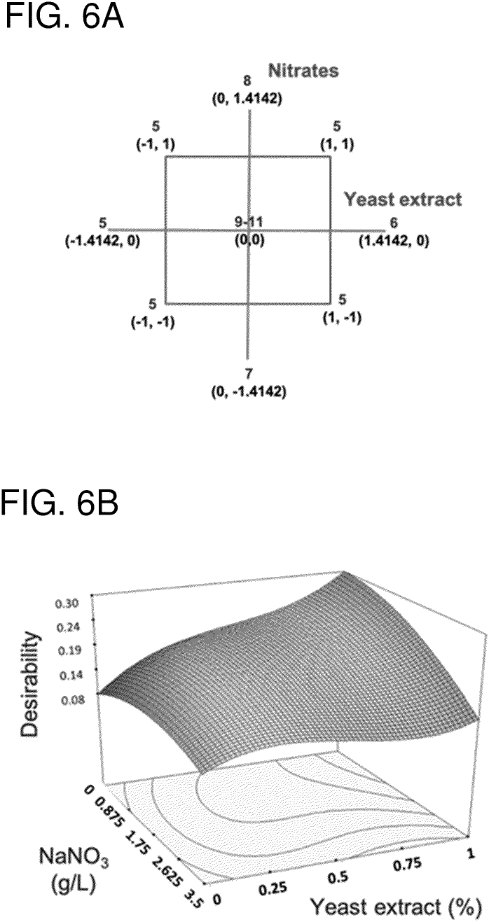

FIG. 6. Optimisation of P. putredinis NO1 growth media. a) A central composite design was used to create a response surface morphology to yeast extract and sodium nitrate concentrations. b) Both cellulase and xylanase production was improved with a high yeast extract and low nitrate concentrations;

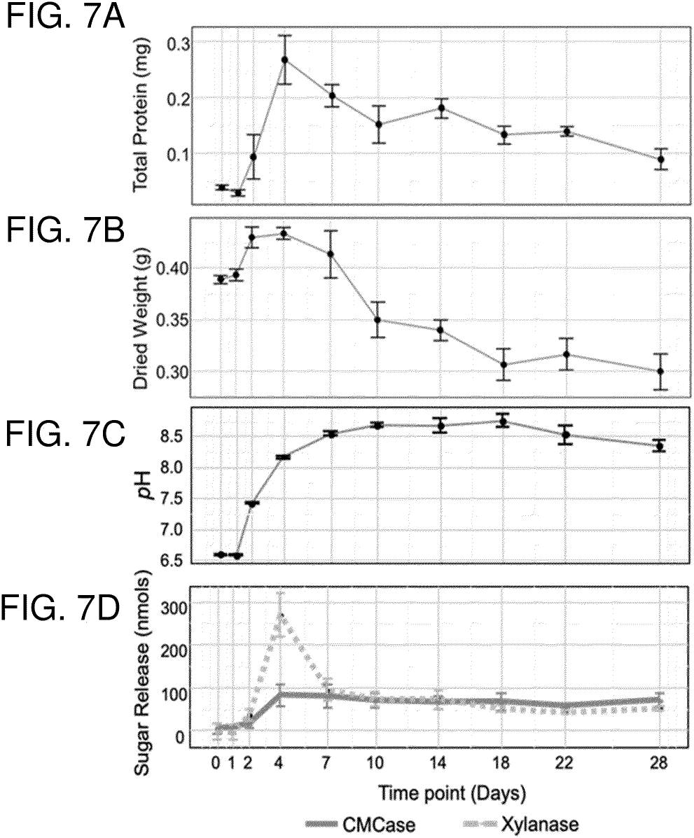

FIG. 7. Growth of P. putredinis NO1 on wheat straw over a period of one month. a) Growth of P. putredinis NO1 on wheat straw estimated by the total protein present in the culture and b) the dried weight of the total biomass within the culture. c) The pH of the culture was also monitored alongside d the release of sugar after 1 h from 10% supernatant loading on carboxymethylcellulose and beechwood xylan;

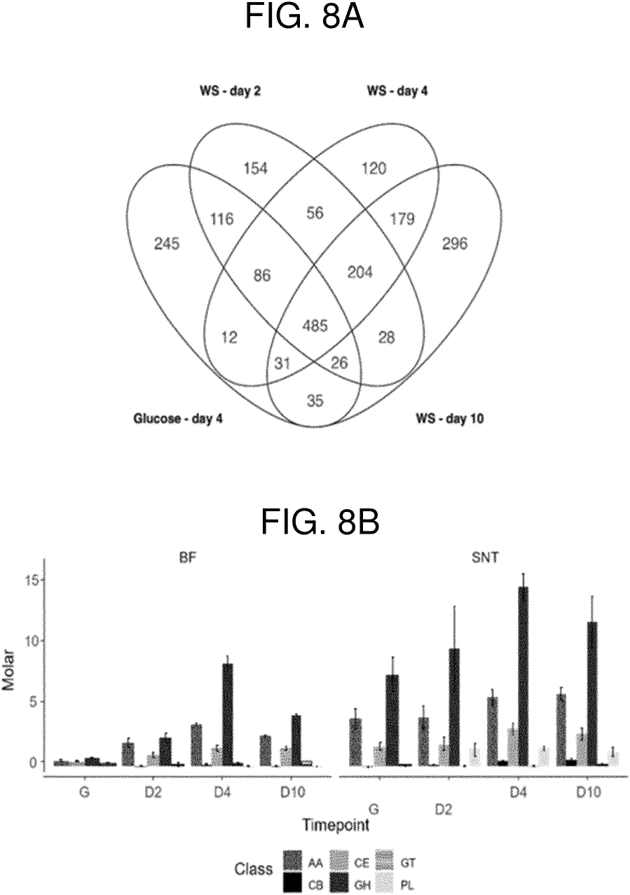

FIG. 8. Proteomics of P. putredinis NO1 grown on wheat straw. a) Total proteins recovered from P. putredinis NO1 exoproteome across timepoints. b) Total molar percentage of CAZy class across timepoints in the biotin labelled protein sample and supernatant;

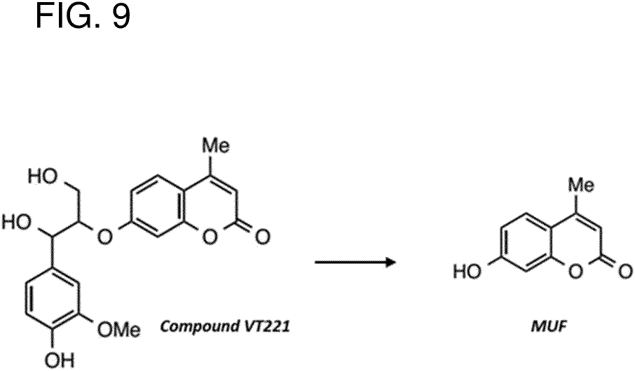

FIG. 9. GGβ4MU β-etherase assay. Under the action of a β-etherase the 4—O—β-ether linkage is cleaved liberating the product MUF. Upon excitement at 372 nm MUF will fluoresce at 445 nm;

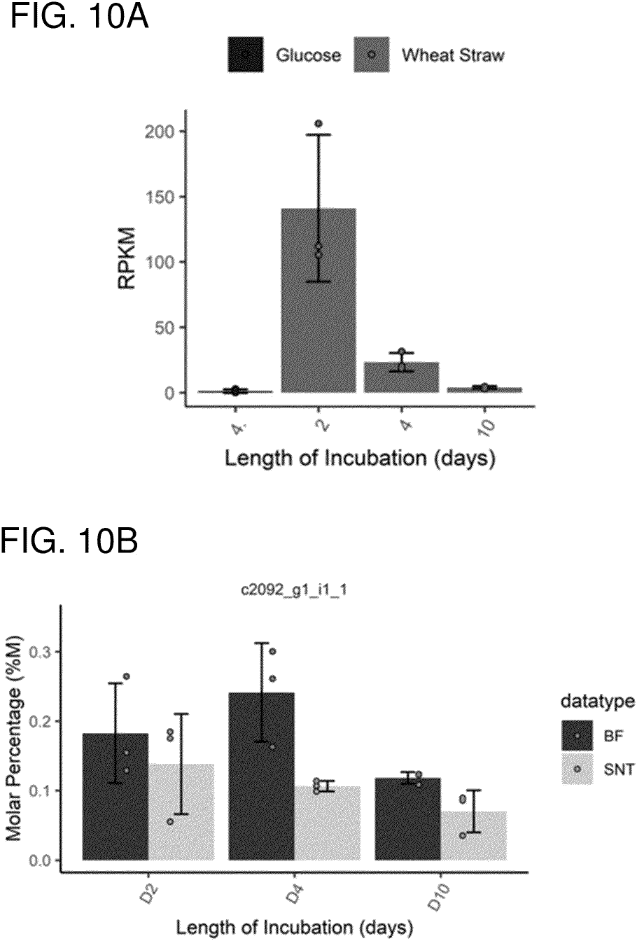

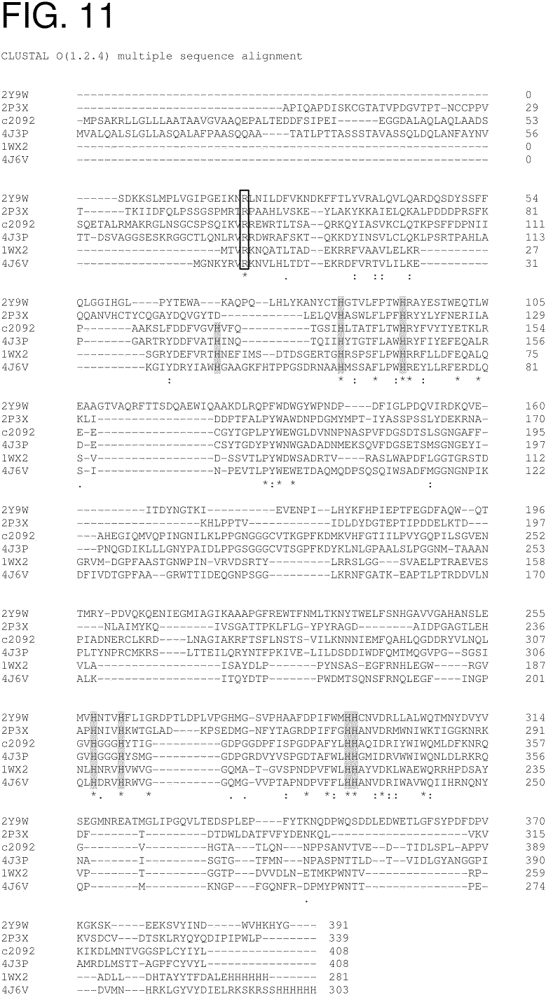

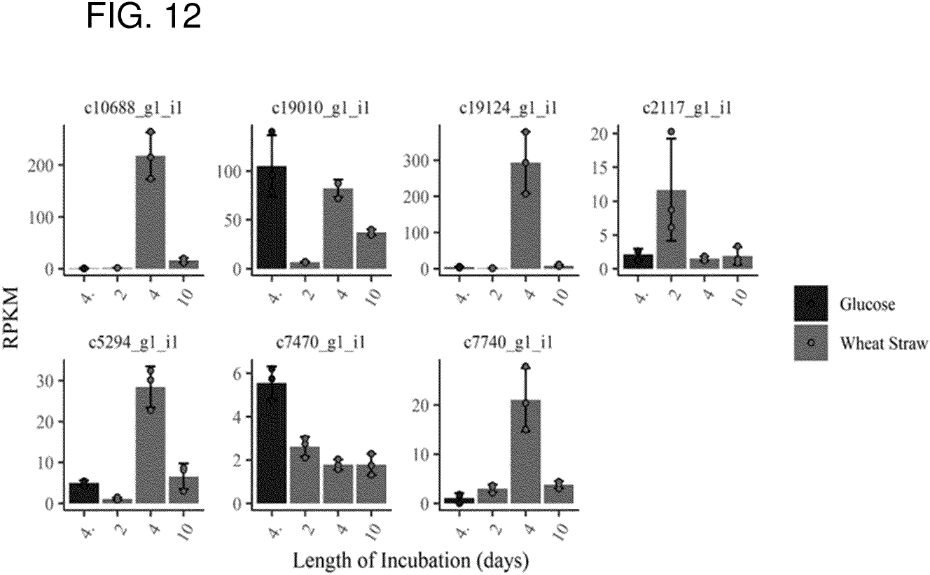

FIG. 10. c2092_g1_i1 abundance within the a) transcriptomic and b) proteomic libraries. Circles represent sample values of biological replicates (N=3), and error bars ± SD of the mean;

FIG. 11. Alignment of β-etherase amino acid sequence (c2092) with structurally related enzymes. Alignment with 2Y9W; tyrosinase from Agaricus bisporus (common mushroom), 2P3X; Vitis vinifera Polyphenol Oxidase, 4J3P; catechol oxidase Aspergillus oryzae, 1WX2; Streptomyces castaneoglobisporus tyrosinase, 4J6V; Bacillus megaterium N205D tyrosinase. Identical amino acids are indicated by asterisks and amino acids similarity by dots. The conserved N-terminal arginine residue is circled ; copper-binding regions are highlighted;

FIG. 12. Reads per kilobase per million (RPKM) of contigs identified as sharing significant similarity of the putative β-etherase. Reads with a similarity identity of over 30% to c2092 were considered as displaying significant homology. Circles represent sample values of biological replicates (N=3), and error bars ± SD of the mean;

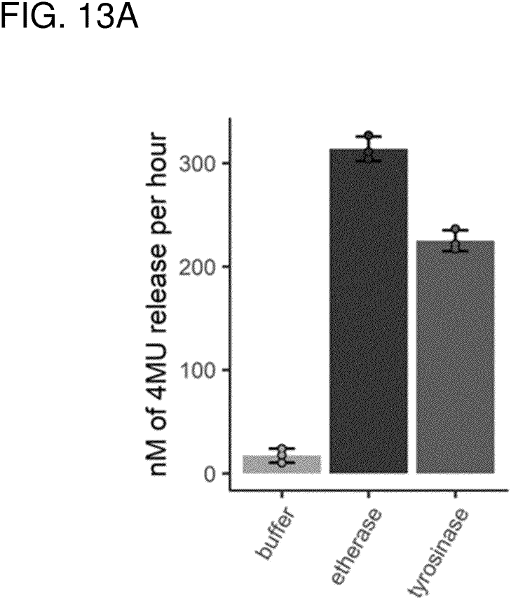

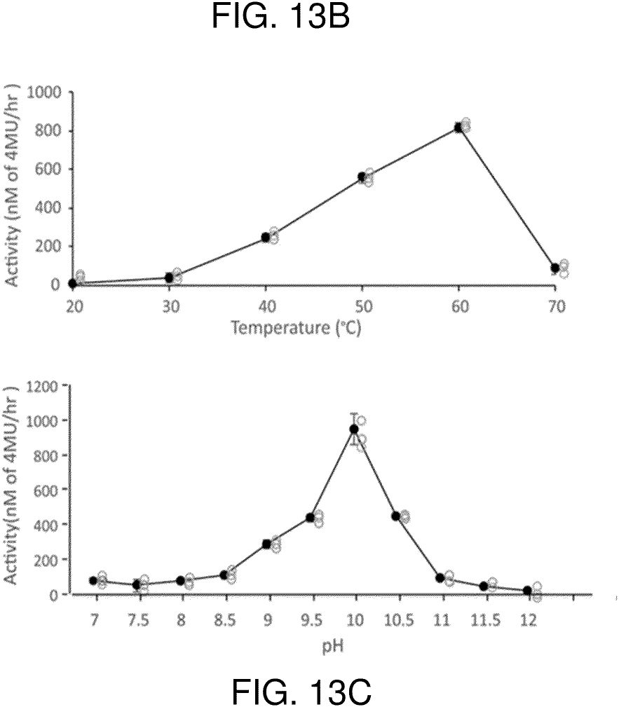

FIG. 13. Activity of the putative β-etherase against the synthetic substrate GGβ4MU. a) Fluorescence activity of purified β-etherase against tyrosinase and buffer control reaction. b-c) optimum temperature and pH for purified β-etherase as assessed by GGβ4MU assay. Circles represent sample values, and bars sample mean ± SD, N=3;

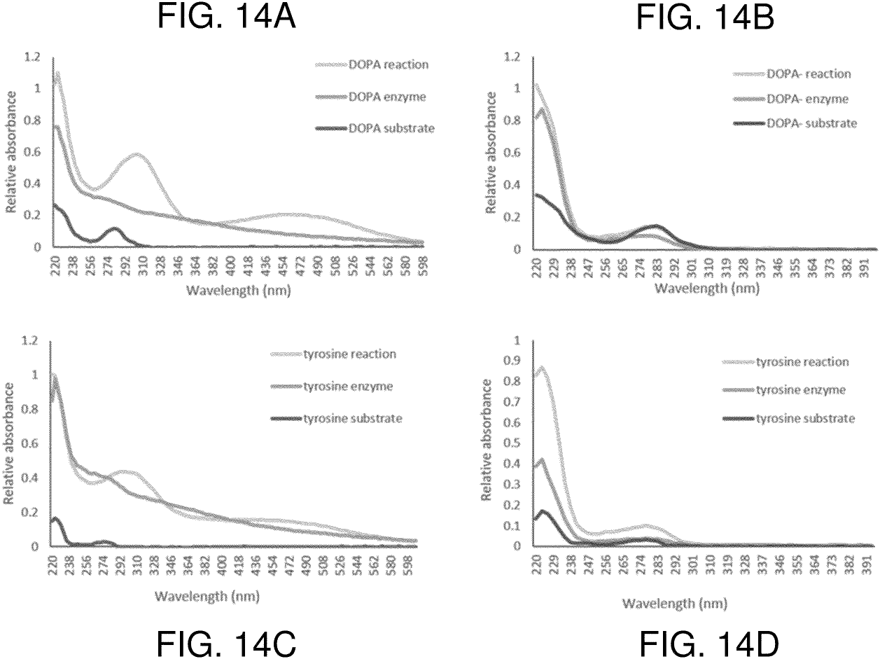

FIG. 14. UV spectrum showing oxidase activity of β-etherase against tyrosinase substrates. Either was incubated in 50 mM Tris pH 8.5 at room temperature with 1 mM of substrate against enzyme only or substrate only as controls, a) _{L}-DOPA reaction with tyrosinase, b) _{L}-DOPA reaction with β-etherase, c) tyrosine reaction with tyrosinase, d) tyrosine reaction with β-etherase;

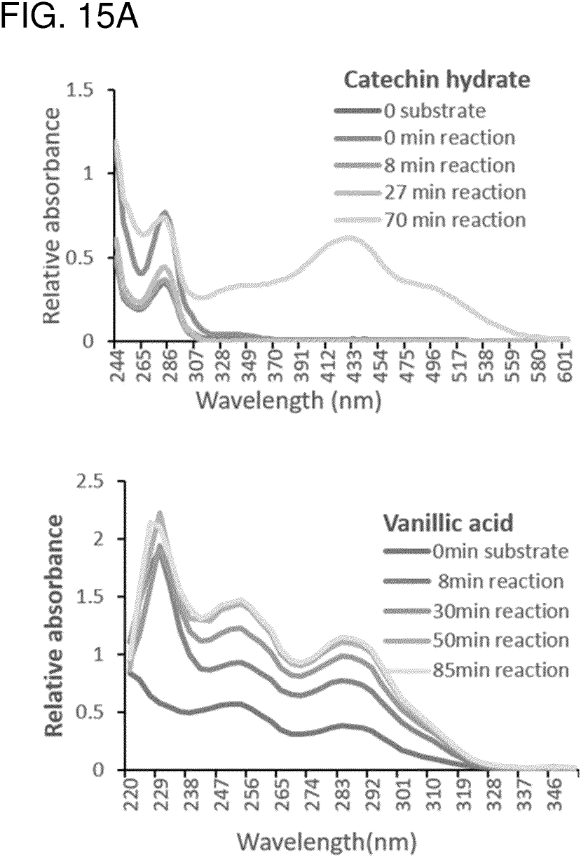

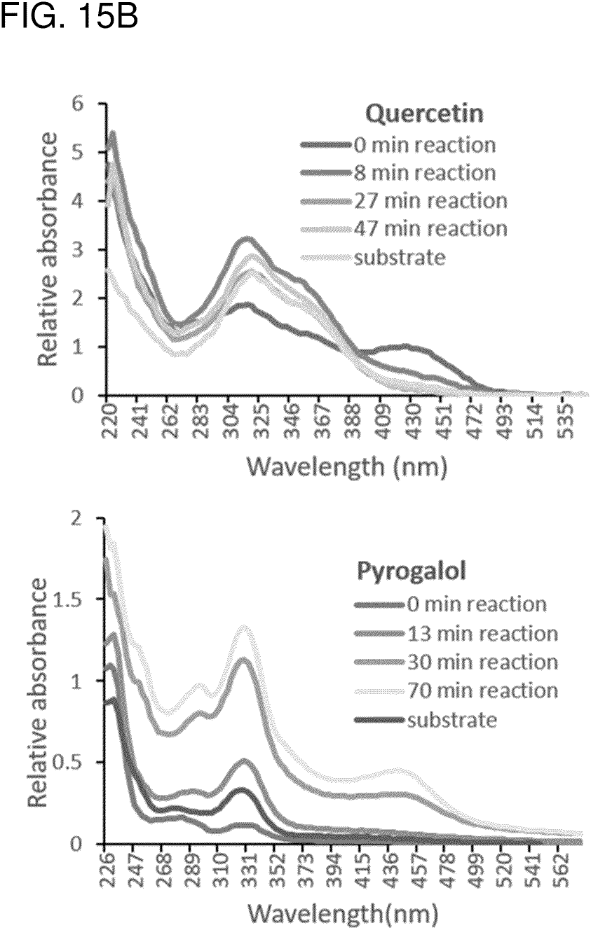

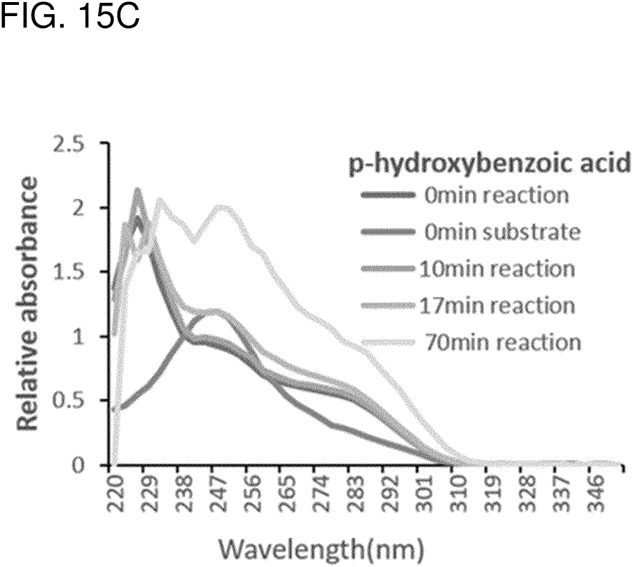

FIG. 15. UV spectrum showing oxidase activity of β-etherase against different phenolic compounds. 1 mg/mL of the enzyme was incubated in 50 mM Tris pH 8.5 at room temperature with 1 mM of either catechin hydrate, pyrogallol, vanillic acid, p-hydroxybenzoic acid or quercetin. UV-Vis spectra were recorded at regular intervals; and

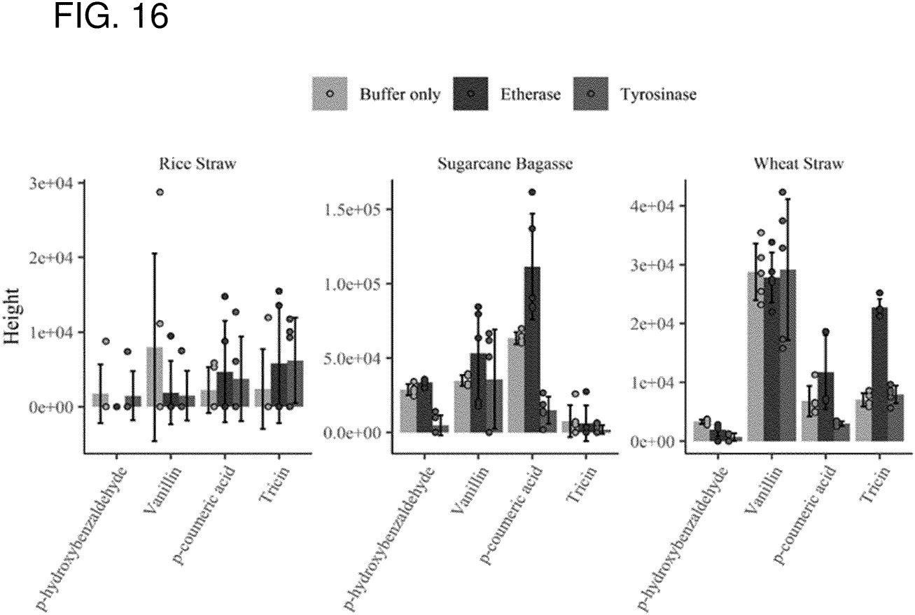

FIG. 16. Release of products from lignocellulosic substrates after incubation with β-etherase, mushroom tyrosinase and buffer only. Reactions were performed at physiological -pH 8.5 & 30° C. prior to the reaction products being extracted from the reaction supernatant using ethyl acetate and analysed with high-performance liquid-chromatography. Circles represent the individual sample values (N=5), and error bars ± SD of the mean.

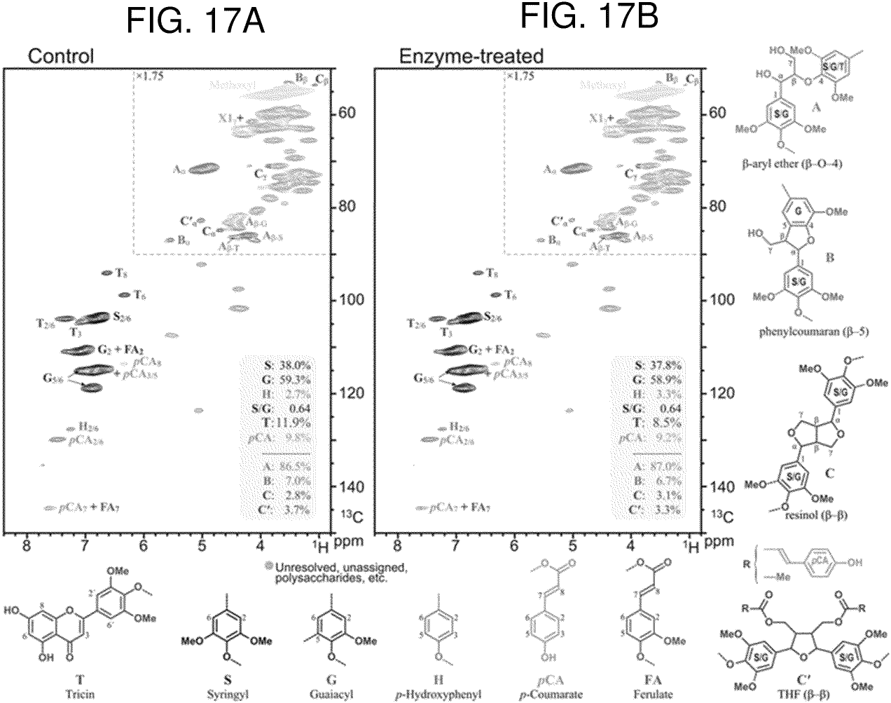

FIG. 17. Lignin aromatic and side-chain region of 2D HSQC NMR spectra (DMSO-d₆:pyridine-d₅, 4:1, v/v) of enzyme lignins (EL) from (A) the wheat control, and (B) the enzyme-treated wheat. Signal assignments in the spectra correspond to the chemical structures of the lignin monomeric subunits shown (S) syringyl, (G) guaiacyl, (H) p-hydroxyphenyl, (T) tricin, (pCA) p-Coumarate, (A) β-aryl ether (β—O—4), (B) phenylcoumaran (β-5), (C) resinol (β-β).

The quantification values shown in the table are for relative comparisons of the lignin components determined from NMR contour volume-integrals based on S + G + H = 100%. The pCA and T units are lignin appendages; their levels were estimated and expressed based on the total lignin (S + G + H). Assignments are from papers noted in the Experimental Section, along with the newly Aβ-T assignment (80). Note that, to allow the crucial lignin side-chain contours to be more clearly seen, the boxed lignin side-chain region was vertically scaled by ~1.75×.

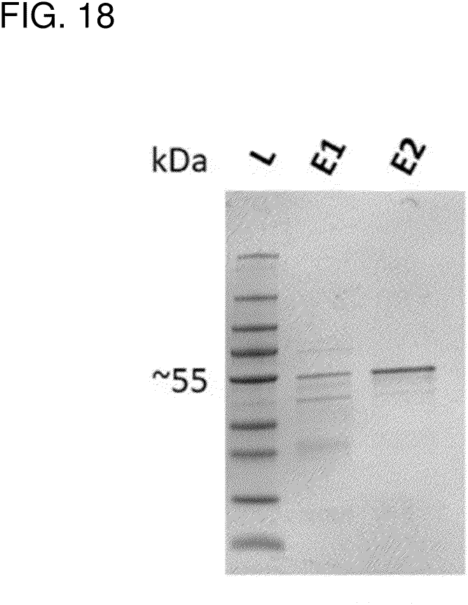

FIG. 18. SDS-PAGE after denaturation, purification and refolding. L is protein marker -Thermo Scientific™ PageRuler™ Plus Prestained Protein Ladder, 10 to 250 kDa. E1 is protein purified in the absence of CuSO₄, and E2 was purified with CuSO₄ present in the refolding buffer.

TABLE 1

Proteins showing homology to the putative β-etherase within P. putredinis NO1 transcriptome. BLASTp searches were performed on the c2092_g1_i1 sequence (SEQ ID NO 9) against the assembled P. putredinis NO1 transcriptome

| SEQ ID | evalue | pident | length | bitscore | Similarity% | Similarity |
|---|---|---|---|---|---|---|
| c19124_g1_i1_4 (SEQ IQ NO 10) | 9.4E-111 | 43.796 | 411 | 330 | 0.608 | 256/421 |
| c7740_g1_i1_6 (SEQ ID NO 11) | 8.17E-77 | 38.482 | 382 | 243 | 0.508 | 23/439 |
| c10688_g1_i1_2 (SEQ ID NO 12) | 1.72E-74 | 40.395 | 354 | 236 | 0.52 | 226/435 |
| c5294_g1_i1_3 (SEQ ID NO 13) | 1.65E-71 | 37.366 | 372 | 229 | 0.52 | 223/429 |
| c2117_g1_i1_2 (SEQ ID NO 14) | 2.9E-57 | 36.936 | 349 | 191 | 0.422 | 184/436 |
| c19010_g1_i1_4 (SEQ ID NO 15) | 2.94E-32 | 29.254 | 335 | 125 | 0.325 | 164/505 |
| c7470_g1_i1_2 (SEQ ID NO 16) | 2.25E-26 | 23.37 | 368 | 108 | 0.376 | 169/449 |

TABLE 2

Proteins with homology to the β-etherase within NCBI non-redundant database. BLASTp searches were performed on the c2092_g1_i1 sequence against the non-redundant protein database held by NCBI. Results were filtered to >50 % identity

| | Description | Max Score | Total Score | Query Cover | E value | Percent identity |
|---|---|---|---|---|---|---|
| gb\|PKS12997.1\| | hypothetical protein jhhlp_000338 [Lomentospora prolifcans] | 713 | 713 | 100% | 0.0 | 87.50% |
| ref\|XP_016642676.1\| | Tyrosinase central domain protein [Scedosporium apiospermum] | 674 | 674 | 100% | 0.0 | 82.40% |
| gb\|TPX10091.1\| | hypothetical protein E0L32_001288 [Phialemoniopsis curvata] | 572 | 572 | 93% | 0.0 | 67.19% |
| gb\|ELA32929.1\| | tyrosinase central domain protein [Colletotrichum fructicola Nara gc5] | 506 | 506 | 99% | 7e-176 | 57.95% |
| gb\|KZL67883.1\| | tyrosinase central domain-containing protein [Colletotrichum tofieldiae] | 501 | 501 | 97% | 8e-174 | 58.90% |
| gb\|EQB58959.1\| | hvpothetical protein CGLO_00722 [Colletotrichum gloeosporioides Cg-14] | 497 | 497 | 92% | 3e-172 | 59.89% |
| gb\|KZL82263.1\| | tyrosinase central domain-containing protein [Colletotrichum incamum] | 496 | 496 | 97% | 3e-172 | 58.15% |
| gb\|KXH49404.1\| | tyrosinase central domain-containing protein [Colletotrichum nymphaeae SA-01] | 486 | 486 | 99% | 2e-168 | 55.88% |
| gb\|KXH49404.1\| | tyrosinase central domain-containing protein [Colletotrichum simmondsii] | 485 | 485 | 99% | 1e-167 | 55.64% |
| gb\|OLN85731.1\| | Grixazone synthase 2 [Colletotrichum chlorophyti] | 484 | 484 | 92% | 3e-167 | 58.99% |
| ref\|XP_018157362.1\| | tyrosinase central domain-containing protein [Colletotrichum higginsiamamIMI 349063] | 481 | 481 | 92% | 4e-166 | 59.37% |
| gb\|EXF76797.1\| | tyrosinase central domain-containing protein [Colletotrichum fioriniae PJ7] | 479 | 479 | 99% | 2e-165 | 55.15% |
| gb\|TDZ75107.1 | tyrosinase-like protein orsC {colletotrichum trifolii] | 476 | 476 | 92% | 4e-164 | 59.95% |
| gb\|TKW48599.1\| | hypothetical protein CTA1_467 [Colletotrichum tanaceti] | 473 | 473 | 92% | 7e-163 | 58.42% |
| gb\|TDZ15437.1\| | tyrosinase-like protein orsC [colletotrichum orbiculare MAFF 240422] | 470 | 470 | 92% | 4e-162 | 60.48% |
| ref\|XP_001227696.2\| | hypothetical protein CHGG 09769 [Chaetomium globosum CBS 148.51] | 469 | 469 | 100% | 2e-161 | 55.50% |
| gb\|TDZ29471.1\| | Tyrosinase-like protein orsC [colletotrichum spinosum] | 460 | 460 | 92% | 2e-157 | 57.00% |
| ref\|XP_022470530.1\| | tyrosinase central domain-containing protein [Colletotrichum orchidophilum] | 458 | 458 | 99% | 2e-157 | 54.66% |
| gb\|OIW32989.1 | tyrosinase central domain-containing protein [Coniochaeta ligniaria NRRL30616 | 447 | 447 | 92% | 5e-153 | 53.79% |
| gb\|KXH30586.1\| | tyrosinase central domain-containing protein [Colletotrichum salicis] | 447 | 447 | 97% | 3e-152 | 54.02% |
| gb\|RKU41032.1\| | hypothetical potein DL546 002981 [Coniochaeta pulveracea] | 442 | 442 | 99% | 5e-151 | 51.96% |
| gb\|KZL64229.1\| | tyrosinase central domain-containing protein [Colletotrichum incanum] | 434 | 434 | 92% | 4e-145 | 55.17% |
| gb\|TEA15757.1\| | Tyrosinase-like protein orsC [Colletotrichum sidae] | 427 | 427 | 92% | 6e-145 | 55.00% |
| gb\|OHW92206.1\| | tyrosinase central domain-containing protein [Colletotrichum incanum] | 420 | 420 | 84% | 5e-143 | 57.73% |
| ref\|XP_01816298.1\| | Tyrosinase central domain-containing protein [Colletotrichum higginsianum IMI 349063] | 425 | 425 | 92% | 1e-142 | 54.38% |
| gb\|TID02585.1\| | Tyrosinase ustQ [Colletotrichum higginsianum] | 425 | 425 | 92% | 1e-142 | 54.38% |
| gb\|OLN83361.1\| | Tyrosinase 2 [Colletotrichum chlorophyti] | 417 | 417 | 92% | 5e-141 | 51.97% |
| emb\|CCF32411.1\| | hypothetical protein CH063 04807 [Colletotrichum higginsianum | 412 | 412 | 84% | 7e-140 | 56.85% |
| gb\|KZL72889.1\| | tyrosinase-like protein [Colletotrichum tofieldiae] | 412 | 412 | 84% | 7e-140 | 57.14% |
| gb\|TKW50870.1\| | hypothetical protein CTA1 3684 [Colletotrichum tanaceti] | 419 | 419 | 92% | 7e-140 | 52.39% |
| gb\|KDN70624.1\| | hypothetical protein CSUB01 04485 [Colletotrichum sublineola] | 417 | 417 | 92% | 1e-139 | 53.58% |
| gb\|EXF84421.1\| | hypothetical protein CFIO01_02736 [Colletotrichum fioriniae PJ7] | 409 | 409 | 92% | 1e-136 | 52.22% |
| gb\|XP_003664995.1\| | tyrosinase-like protein [Thermothelmyces thermophilus ATCC 42464] | 404 | 404 | 92% | 3e-136 | 54.09% |
| gb\|TQN72542.1 | Tyrosinase-like protein orsC [Colletotrichum sp. PG-2018a] | 407 | 407 | 89% | 5e-136 | 54.77% |
| ref\|XP_003351009.1\| | uncharacterized protein SMAC 04313 [Sordaria marcrospora k-hell] | 399 | 399 | 97% | 6e-134 | 50.12% |
| ref\|XP_006692366.1\| | hypothetical protein CTHT 0018720 [Chaetomium thermophilum yar. thermophilum DSM 1495] | 395 | 395 | 89% | 1e-132 | 54.67% |
| gb\|TDZ58291.1\| | Tyrosinase-like protein orsC [Colletotrichum trifolii] | 393 | 393 | 79% | 6e-132 | 57.67% |
| gb\|TDZ23501.1\| | Nitroalkane oxidase [Colletotrichum orbiculare MAFF 240422] | 409 | 409 | 80% | 8e-132 | 57.75% |
| ref\|XP_022471338.1\| | hypothetical protein COR01 10513 [Colletotrichum orchidophilum] | 397 | 397 | 92% | 9e-132 | 50.78% |
| gb\|KXH34366.1\| | hypothetical protein CSIM01 00277 [Colletotrichum simmondsii] | 396 | 396 | 92% | 2e-131 | 50.51% |
| gb\|KXH69104.1\| | hypothetical protein CSAL01 01466 [Colletotrichum salicis] | 389 | 389 | 81% | 3e-129 | 56.19% |
| ref\|XP_008090963.1\| | hypothetical protein GLRG 02114 [Colletotrichum graminicola M1.001 | 378 | 378 | 79% | 2e-126 | 56.44% |
| ref\|XP_001227853.1\| | hypothetical protein CHGG 09926 [Chaetomium globosum CBS 148.51] | 373 | 373 | 92% | 5e-124 | 50.00% |
| gb\|TDZ28941.1\| | Tyrosinase-like protein orsC [Colletotrichum spinosum] | 371 | 371 | 73% | 2e-122 | 58.14% |
| gb\|ELA37064.1\| | hypothetical protein CGGC5 3508 [Colletotrichum fructicola Nara gc5] | 364 | 364 | 72% | 1e-121 | 59.52% |
| ref\|XP_007911158.1 | putative tyrosinase-like protein [Phaeoacremonium minimum UCRPA7] | 363 | 363 | 68% | 2e-121 | 59.22% |
| gb\|EQB52888.1\| | hypothetical protein CGLO 07432 [Colletotrichum gloeosporioides Cg-14] | 361 | 361 | 72% | 2e-120 | 59.86% |
| gb\|TEA10724.1\| | Nitroalkane oxidase [Colletotrichum sidae] | 373 | 373 | 73% | 4e-118 | 58.33% |
| ref\|XP_024731024.1\| | putative tyrosinase [Meliniomyces bicolor E] | 331 | 331 | 79% | 2e-108 | 51.38% |
| emb\|CDP29730.1\| | Putative tyrosinase [Podospora anserina S mat+ | 326 | 326 | 81% | 4e-106 | 50.15% |
| emb\|VBB81548.1\| | Putative tyrosinase [Podospora comtat] | 326 | 326 | 81% | 5e-106 | 50.15% |
| ref\|XP_001273822.1\| | tyrosinase, putative [Aspergillus clavatus NRRL 1] | 326 | 326 | 83% | 2e-105 | 50.00% |
| ref\|XP_001905273.1\| | uncharacterized protein PODANS 5 7820 [Podospora anserina S mat+] | 323 | 232 | 80% | 3e-105 | 50.00% |
| gb\|PGH18781.1\| | hypothetical protein AJ79_00194 [Helicocarpus griseus UAMH5409] | 325 | 325 | 83% | 5e-105 | 50.15% |
| gb\|PBP21500.1\| | hypothetical protein BUE80 DR007716 [Diplocarpon rosae] | 278 | 278 | 68% | 4e-88 | 50.17% |

TABLE 3

Purification of β-etherase. The heterologously expressed protein was purified using anion-exchange (Q) and size-exclusion chromatography (S.E). Protein concentration and VT221 activity was calculated after each purification step

| Purification steps | Total Protein mg | Activity (mU) (nmol/mg/hr) | Specific (U/mg) | Yield (%) | Purification fold |
|---|---|---|---|---|---|
| Culture filrate | 1024 | 7500 | 7.32 | 100 | 1 |
| Q | 29.25 | 2600 | 88 | 34.67 | 12 |
| S.E | 14 | 1950 | 139 | 26 | 19 |

TABLE 4

β-etherase substrate specificity

| | | |
|---|---|---|
| Substrate | Etherase reactivity | Tyrosinase reactivity |
| Tyrosine methyl ester | — | + |
| L-Dopa (3,4-dihydroxy-L-phenylalanine) | — | + |
| Dopamine hydrochloride | — | + |
| Caffeic acid (catechol oxidase substrate) | — | + |
| 4-Methly-catechol (catechol oxidase substrate) | — | + |
| Tyrosol (catechol oxidase substrate) | — | — |
| Tannic acid | — | — |
| (+)-Catechin hydrate | + | + |
| Pyrogallol | + | + |
| 4-Hydroxybenzoic acid | + | — |
| Quercetin | + | — |
| Vanillic acid | + | — |

### MATERIAL AND METHODS

### Wheat Straw Degradation in Shake-Flasks Inoculated with Compost

Two-liter shake flasks, containing 1 L minimal media and 5% (w/v) milled wheat straw, were inoculated with 1% (w/v) compost. The inoculum was collected from composting wheat straw that had been developed over the period of a year and watered at regular intervals. The inoculum was prepared by blending until homogenized and used on the day of preparation. The minimal media was based on Aspergillus niger minimal media and contained KCI 0.52 g/L, KH₂PO₄ 0.815 g/L, K₂HPO₄ 1.045 g/L, MgSO₄ 1.35 g/L, NaNO₃ 1.75 g/L, Hutner’s trace elements (Na₂EDTA·2H₂O 50 g/L, ZnSO₄·7H₂O 22 g/L, H₃BO₃ 11.4 g/L, MnCl₂·4H₂O 0.506 g/L, FeSO₄·7H₂O 0.4499 g/L, CoCl₂·6H₂O 0.161 g/L, CuSO₄₋5H₂O 0.157 g/L, (NH₄)₆Mo₇O₂₄·4H₂O 0.110 g/L). These flasks were incubated at 30° C. and shaken at 150 rpm. Aliquots (10 mL) containing both the solid and liquid fractions were aseptically collected weekly for eight weeks. The samples were then serially diluted with x1 phosphate-buffered saline to concentrations ranging between 10⁻¹ and 10⁻⁷. From these dilutions 100 µL samples were used to create spread plates on both nutrient agar (NA) and potato dextrose agar (PDA), in order to culture strains from the composting environment.

### Targeted Amplicon Sequencing of 16S and ITS Region

Genomic DNA was harvested from the compost cultures using a modified CTAB protocol adapted for use on materials with high phenolic contents. From the composting shake flask, 20 mL aliquots were harvested weekly. The biomass was separated from the liquid fraction by centrifugation performed at 4000 g at 4° C., and 0.5 g of biomass removed to a 2 mL screw-cap tube. To this 500 µL of cetyltrimethylammonium bromide (CTAB) buffer (2% (w/v) CTAB 100 mM Tris-HCI (pH 8.0), 20 mM EDTA (pH 8.0), 2 M NaCl, 2% (w/v) polyvinylpyrrolidone (Mr 40.000), 5% 2-mercaptoethanol (v/v), 10 mM ammonium acetate, was added along with 0.5 g of zirconia beads and 0.5 mL of phenol: chloroform: isoamyl alcohol (25: 24: 1, pH 8.0), before briefly vortexing. The material was then bead-beaten using a TissueLyser II (Qiagen) for 5 min at speed 28/s. A modified phenol-chloroform method was used to extract DNA after cell lysis. The sample was spun for 5 min at max speed to achieve separation of the phases before the aqueous layer was removed to a fresh 2 mL Eppendorf tube. To the aqueous phase chloroform: isoamyl alcohol (21:1) was added, and this was spun and the aqueous phase transferred to a fresh tube, to remove any remaining phenolics. To precipitate the DNA within the sample, an equal volume of ice-cold 100% isopropanol was added and incubated for 1 h. DNA was pelleted by centrifugation at 13,000 rpm for 10 min, and supernatant was removed without disturbing the pellet. The pellet was then washed with 80% ethanol, before being resuspended in DNAse-free water.

Regions for amplicon sequencing were amplified using Phusion® High-Fidelity DNA Polymerase (Finnzymes OY, Finland) as per manufactures instructions before being purified with Agencourt AMPure XP (Beckman Coulter), and sequenced at the Biorenewables Development Centre (BDC), York, U.K. using an Ion Torrent platform. The primers pairs, for ITS and 16S sequencing, were as follows; ITS1 Fw - TCCGTAGGTGAACCTGCGG (SEQ ID NO 34), Rv - CGCTGCGTTCTTCATCG (SEQ ID NO 35), 16S Fw -AYTGGGYDTAAAGNG (SEQ ID NO 36), Rv-TACNVGGGTATCTAATCC(SEQ ID NO 37). Ribosomal DNA sequence data generated via targeted amplicon sequencing was analyzed using the open-access software Qiime on the University of York’s Technology Facilities linux server. ⁵⁷ Each fastq file generated from the IonTorrent platform was first demultiplexed and then converted into both fasta and qual file types using Qiimes python script convert_fastaqual_fastq.py. To remove the primer sequences from the reads, the script split_libraries.py was used along with a mapping file generated as per Qiimes instructions. Low-quality reads were removed by filtering out reads under 180 bp long and those without recognizable primers. The orientation of the sequences was then corrected based on the primer location. Operational taxonomic units (OTUs) were then created from the fasta files. These files were picked using the open-reference OTU picking process. To perform this, the script pick_open_reference_otus.py was used. This step also includes taxonomy assignment, sequence alignment, and tree building steps. For the taxonomy assignments of bacterial sequences the default reference database was used, (greengenes gg_13_8 97_otus database),^{58,59} and for the fungal ITS sequences the UNITE (alpha release 12_11) database was used.⁶⁰

### Central Composite Design for Media Optimisation

Media was optimized using a central composite design with rotation.⁶¹ It was optimized for the production of both cellulase and xylanase enzymes after seven days on 1.5% wheat straw and minimal media, as assessed by measuring reducing sugar release after incubation on CMC and xylan. The concentrations of both sodium nitrate and yeast extract were varied as part of the optimization. The sodium nitrate concentration was varied between 0 g/L and 3.5 g/L, and yeast extract was varied between 0% and 1% (w/v). Statistica 6.0 software was used to create the experimental design and analyze the results.

The optimized media for P. putredinis NO1 growth consisted of yeast extract 8.55 g/L, KCI 0.52 g/L, KH₂PO₄ 0.815 g/L, K₂HPO₄ 1.045 g/L, MgSO₄ 1.35 g/L, NaNO₃ 1.75 g/L and Hutner’s trace elements.

### Characterization of P. Putredinis NO1 Growth on Wheat Straw

Growth of P. putredinis NO1 was assessed using the dried weight of the biomass present within the culture. Cultures were transferred to pre-weighed and freeze-dried falcon tubes and chilled for 5 min. They were then centrifuged at 4,500 rpm, and the supernatant removed. The biomass was gently rinsed with x1 PBS and tubes were flash-frozen in liquid nitrogen and lyophilized. Each tube was then re-weighed to calculate the dry weight of the biomass present. The total protein content of the cultures was used as an indicator of growth on insoluble materials such as wheat straw. Total protein was extracted by boiling 100 µg of freeze-dried biomass in 1 mL of 0.2% (w/v) sodium dodecyl sulfate, for 5 min to lyse all cells present. Protein was then collected by centrifugation at 14,000 rpm and the supernatant collected into a fresh 50 mL falcon tube. This was repeated three times, without heating, and with vigorous vortexing between each centrifuge step to wash the biomass of any remaining protein. Extracted protein was precipitated with five volumes of ice-cold acetone overnight at -20° C., before being centrifuged at 4500 rpm and the resulting pellet washed with 80% (v/v) ice-cold ethanol. The ethanol-protein mix was then centrifuged again, and the supernatant removed and the pellet air-dried. The protein was then solubilized in 3 mL of H₂O and quantified using the Bradford assay. The ability of an enzyme to cleave polysaccharides and produce products with reducing ends was assessed at each timepoint by incubating 10 µL of cultural supernatant with the 2% (w/v) of either carboxymethylcellulose (CMC) or xylan (beechwood) in 200 µL of 50 mM sodium phosphate at 6.8 and 30° C. Before and after incubation 10 µL aliquots mixed with p-hydroxybenzoic acid hydrazide (PAHBAH), heated to 70° C. for 10 min, and color change detected at 415 nm using a microtitre Tecan Safire2 plate reader.⁶² A stock solution of the appropriate monosaccharide was assayed to obtain a standard curve for quantification of sugar release.

### RNA Extraction from P Putredinis NO1 Sp

Cultures of P. putredinis NO1 were established in 200 mL shake flasks, containing 20 mL of the optimized growth media and either 1.5% wheat straw or 0.5% glucose. These were incubated at 30° C. with shaking at 180 rpm. To control for varying amounts of cell growth, aliquots of either 0.5 g, 0.3 g and 0.1 g of biomass from the wheat straw cultures were weighed into 2 mL screw-cap tubes that contained 3×3 mm tungsten carbide beads and 1 mL Trizol (Life Technologies). The cells were then disrupted in a TissueLyser II (Qiagen) for either 2×2 min or 2×5 min at 28/s, dependent on the stage of growth. Total RNA was then extracted with the standard Trizol method as per manufacturer’s instructions and extracted RNA was resuspended in 50 µL of nuclease-free water. The quality of RNA was assessed by visualization on agarose gels. To obtain enough RNA for processing six technical replicates were performed for each biological replicate. These were stored at -80° C. after being flash-frozen in liquid nitrogen before further processing could occur. The RNA samples were treated for DNA contamination with RTS DNase kits (Mobio) using standard methods described by the manufacturers. The samples were then cleaned with ZymoResearch RNA Clean &

Concentrator™ 5 kits, using the manufacturer’s protocol to separate small and large RNA fragments into different fractions. RNA fragments greater than 200 nt were elution into 50 µL of RNase-free water before RNA concentration, and quality was evaluated with the 2200 TapeStation (Aligent). Once total RNA of a suitable quantity and quality was obtained, samples could be enriched for messenger RNA (mRNA). This was performed using Ribo-Zero™ Magnetic Epidemiology rRNA removal kit (RZE1224/MRZ11124C; Illumina) according to the manufacturer’s protocol.

### RNA Sequencing

The Genome Analysis Centre (TGAC), Norwich, U.K, performed the RNA sequencing on an Illumina HiSeq platform. As per the requirements of the sequencing service, 100 ng of enriched mRNA was provided for each sample. From the proved mRNA, cDNA libraries were constructed using the adapted TruSeq RNA v2 protocol (Illumina 15026495 Rev.B). Libraries were then normalized using elution buffer (Qiagen) and pooled in equimolar amounts into one final 12 nM pool. These were then diluted to a final concentration of 10 pM, spiked with 1% PhiX and loaded onto the Illumina cBotTemplate, for hybridization and first extension, using the TruSeq Rapid PE Cluster Kit v1 before the flow cell was transferred onto the Illumina HiSeq2500. Here, the remainder of the clustering process was conducted, and the library pool was run in a single lane for 100 cycles of each paired-end read before samples were demultiplexed. One base-pair mismatch per library was allowed, and reads were converted to FASTq. The raw data was subject to rRNA removal by catching the remaining paired reads after mapping to a modified rRNA_115_tax_silva_v1.0 ribosomal set, using BOWTIE2. The reads were further trimmed to remove adaptor sequences with the ngsShoRT_2.1 method, and libraries were pooled before being assembled by Trinity Software to obtain 37,720 contigs. Then, using this assembly as a reference, the original (unprocessed) individual libraries were mapped and the number of reads counted for each contig. Counts per million (CPM) were converted to reads per kilobase of exon per million reads mapped (RPKM) to normalize for both the depth of sequencing achieved in each sample and length of the contig.

Emboss GETORF (http://www.bioinformatics.nl/cgi-bin/emboss/getorf) was used to generate putative protein-coding sequences by translating all regions over 300 bp between potential start and stop codons. Putative open reading frames (ORFs) were searched against the NCBI non-redundant protein database and KOG database using BLASTp, and Pfam and dbCAN databases using HMMER3.(45, 81, 82) Local BLAST searches using unique were performed using BLAST+ 2.3.0.(65, 64) Signal peptides were predicted from ORFs using SignalP 4.0.(66, 67)

### Protein Extraction

Supernatant proteins were harvested by collecting samples (20 mL) from the culture supernatant of P. putredinis NO1 and precipitated in five volumes of ice-cold acetone. The acetone fractions were incubated overnight at -20° C., before being centrifuged at 10,000 xg. The resulting pellet was washed with 80% ice-cold acetone, air-dried and resuspended in 0.5x PBS with 0.1% sodium dodecyl sulfate (SDS). To selectively extract biomass bound proteins, two grams of biomass collected from the fungal cultures was washed twice with ice-cold 0.5x PBS, before being resuspended and mixed for 1 h at 4° C., in 0.5x PBS with 10 mM EZ-linked biotin (Thermo Scientific). The reaction was then quenched for 30 min with 50 mM Tris-HCL, pH 8, and excess biotin was removed by washing twice with ice-cold 0.5 × PBS. Warmed SDS (2% w/v, at 60° C.) was used to extract the proteins. The mixture was incubated at room temperature for 1 h, centrifuged and precipitated with ice-cold acetone as described above. The resulting pellets were solubilized in 1x PBS containing 0.1% SDS then loaded onto streptavidin columns (Thermo Scientific) that had been pre-washed (0.1% SDS 1× PBS). The proteins were then incubated for 1 h on the column at 4° C., and washed with three column volumes of 0.1% SDS 1x PBS, before being incubated overnight with elution buffer (50 mM DTT in 1 × PBS) at 4° C. Proteins were eluted the following day by the addition of 1 mL elution buffer and the resulting fraction collected. The column was incubated for one hour before this was repeated. In total the elution was performed four times. These fractions were then flash-frozen in liquid nitrogen, freeze-dried, resuspended in 2 mL distilled water and desalted using Zeba, 7 K MWCO columns (Thermo Scientific) following manufacturer’s instructions. Both the supernatant and biotin-tagged proteins were stored in 4-12% (w/v) Bis-Tris acrylamide gels. Protein samples were loaded into the gel, separated electrophoresis for 20 min and stained with InstantBlue (Sigma-Aldrich).

### Proteomic LC-MS/MS

LC-MS/MS was performed to identify proteins within both the supernatant and biotin-labelled fractions. Proteins contained within gel slices were washed with 50% (v/v) aqueous acetonitrile containing 25 mM ammonium bicarbonate, then reduced with 10 mM DTE and S-carbamidomethylated with 50 mM iodoacetamide. Gels were then dehydrated with acetonitrile and digested with 0.2 µg trypsin (Promega) in 25 mM ammonium bicarbonate. The digestion was performed overnight at 37° C. Peptides were extracted with 50% (v/v) aqueous acetonitrile, dried in a vacuum concentrate and resuspended in 0.1% (v/v) aqueous trifluoroacetic acid. Peptides were loaded onto a nanoAcquity UPLC system (Waters) equipped with a nanoAcquity Symmetry C18, 5 µm trap (180 µm × 20 mm Waters) and a nanoAcquity HSS T3 1.8 µm C18 capillary column (75 mm × 250 mm, Waters). The trap was washed with 0.1% (v/v) aqueous formic acid at a flow rate of 10 µL min⁻¹, before switching to the capillary column. Peptides were separated using a gradient elution of two solvents, 0.1% (v/v) aqueous formic acid (solvent A) and acetonitrile containing 0.1% (v/v) formic acid (solvent B). The flow rate used was 300 nL min⁻¹, and the column temperature was 60° C. The gradient proceeded linearly from 2% solvent B to 30% over 125 min, then 30-50% over 5 min, before being washed with 95% solvent B for 2.5 min. The column was then re-equilibrated at the initial conditions for 25 min before subsequent injections. The nanoLC system was interfaced with a maXis HD LC-MS/MS System (Bruker Daltonics) with a CaptiveSpray ionization source (Bruker Daltonics). Positive ESI- MS & MS/MS spectra were acquired using AutoMSMS mode. Instrument control, data acquisition and processing were performed using Compass 1.7 software (microTOF control, Hystar and DataAnalysis, Bruker Daltonics). Instrument settings were as follows: ion spray voltage: 1,450 V; dry gas: 3 L min⁻¹; dry gas temperature 150° C.; collision RF: 1,400 Vpp; transfer time: 120 ms; ion acquisition range: m/z 150-2,000. AutoMSMS settings specified: absolute threshold 200 counts, preferred charge states: 2-4 , singly charged ions excluded. Cycle time: 1 s, MS spectra rate: 5 Hz, MS/MS spectra rate: 5 Hz at 2,500 cts increasing to 20 Hz at 250,000 cts or above. Collision energy and isolation width settings were automatically calculated using the AutoMSMS fragmentation table. A single MS/MS spectrum was acquired for each precursor, with dynamic exclusion for 0.8 min unless the precursor intensity increased fourfold.

### Genomic Data Analysis

The raw data was subject to rRNA removal by catching the remaining paired reads after mapping to a modified rRNA_115_tax_silva_v1.0 ribosomal set, using BOWTIE2. The reads were further trimmed to remove adaptor sequences with the ngsShoRT_2.1 method, and libraries were pooled before being assembled by Trinity Software to obtain 37,720 contigs. Then, using this assembly as a reference, the original (unprocessed) individual libraries were mapped and the number of reads counted for each contig. Counts per million (CPM) were converted to reads per kilobase of exon per Million reads mapped (RPKM) to normalize for both the depth of sequencing achieved in each sample and length of the contig. Emboss GETORF (http://www.bioinformatics.nl/cgi-bin/emboss/getorf) was used to generate putative protein-coding sequences in all six reading frames from the transcriptomic libraries by translating regions over 300 bp long between potential start and stop codons. These putative open reading frames (ORFs) were searched against the NCBI non-redundant protein database and KOG database using BLASTp, the Pfam and dbCAN databases using HMMER3.^{45,63} Annotations were subsequently mapped back to the contig from which the ORF originated. Local BLAST searches using unique were performed using BLAST+ 2.3.0.^{64,65} Signal peptides were predicted from ORFs using SignalP 4.0.^{66,67}

### Proteomic Data Analysis

Spectra obtained from the LC-MS/MS analysis were searched against all potential opening reads frames generated from the P. putredinis NO1 transcriptomic library, using Mascot (Matrix Science Ltd., version 2.4). This was locally run through the Bruker ProteinScape interface (version 2.1). Search criteria were specified as follows; the instrument was selected as ESI-QUAD-TOF, trypsin was stated as the digestion enzyme, fixed modifications as carbamidomethyl (C), and variable modifications as oxidation (M). Peptide tolerance was 10 ppm, and MS/MS tolerance 0.1 Da. Results were filtered through ‘Mascot Percolator’ to achieve a global false discovery rate of 1%, as assessed against a decoy database and further adjusted to accept only individual peptides with an expect score of 0.05 or lower. An estimation of relative protein abundance was performed as described by Ishihama,⁶⁸ whereby an exponentially modified Protein Abundance Index (emPAI) is used to estimate the relative abundance of proteins in LC-MS/MS experiments. From this index the molar percentage values could be calculated by normalising individual protein Mascot emPAI values against the sum of all emPAI values for each sample. Protein sequences were retrieved using the R package BioStrings.⁶⁹

### Synthesis of Synthetic Substrate GGβ4MU (7-[2-hydroxy-2-(4-hydroxy-3-methoxyphenyl)-1-(hydroxymethyl)ethoxy]-4-methyl-2H-1-benzopyran-2-one).

The synthetic substrate GGβ4MU was synthesized in 6 steps according to the protocol reported by Weinstein and Gold starting from acetovanillone.⁴⁴ The pure substrate GGβ4MU was obtained as a white solid following purification using plate chromatography on silica-gel (10% v/v MeOH in CH₂Cl₂). The NMR data were in excellent agreement with those previously reported.⁴⁴

### Identification of β-Etherase from Native Supernatant

P. putredinis NO1 was cultivated in medium containing 1.5% wheat straw. The supernatant was filtered, and the protein of interest purified by different purification steps, including ammonium sulfate precipitation (ASP), gel filtration using a superdex 200 (GF) on two different columns and anion-exchange chromatography (AE). Briefly, filtered culture supernatant with 0.1% Tween20 was concentrated in a 50 mL stirred Ultracentrifugation Cell (Millipore Corporation, USA) with a Biomax 30 kDa Ultrafiltration Membrane (Millipore Corporation, USA). Ammonium sulfate was slowly added to the filtered culture supernatant to a concentration of 20% while stirring at 4° C. The solution was centrifuged at 10.000 g for 15 min. The pellet was then resuspended in 2 mL buffer A (50 mM Tris-HCI, 100 mM NaCl, 0.1% Tween 20, pH 8.5). Additional ammonium sulfate was added to the supernatant, following the same procedure as described above, to obtain fractions with 30, 40 and 50% ammonium sulfate. After assessing the fractions with the GGβ4MU assay, samples were purified via gel-filtration on a Superdex-200 (GE Healthcare, US), using the ÄKTA system and 50 mM Tris-HCl, 100 mM NaCl, 0.1% Tween 20, pH 8.5. The most active sample was further purified using anion-exchange chromatography. Anion-exchange chromatography was conducted on a DEAE FF column (GE Healthcare, US) with an increasing salt concentration from 0 to 1 M NaCl in 20 min (5 mL/min). A running buffer of 30 mM Tris-HCI, 0.1% Tween 20, at various pH (7.0/7.4/8.5) was used. The Elution buffer was 30 mM Tris-HCI, 1 M NaCl, 0.1% Tween 20.

### Gene Cloning and Expression

The c2092 gene was codon-optimized for expression in E. coli and synthesized into pET151 vector with N-terminal His-tag by Invitrogen. The expression plasmid was transformed into Arctic Express (DE3) competent cells, and successful transformants were selected on LB media containing ampicillin (100 mg L⁻¹) and gentamycin (10 mg L⁻¹). Auto-induction media was used for protein production. Inoculated cultures were incubated at 30° C. with shaking at 180 rpm until an optical density of 0.6 at 600 nm was reached. Once a suitable cell density was reached flasks, the temperature was reduced to 11° C. for 48 h before harvesting.

### Purification of Recombinant β-Etherase

Cell pellets were collected by centrifugation at 7000 rpm and 4° C. for 15 min, then suspended in 50 mL (50 mM Tris, 1 mm DTT, pH 8.5). Suspended pellets were then sonicated on ice for using a Misonix S-4000 sonicator at 70 kHz for 4 min, using a program of 3 s off followed by 7 s on. After centrifugation at 17,000 rpm for 45 min to remove cell debris, the protein was purified by anion-exchange chromatography facilitated by an ÄKTA purifier UPC10 with UNICORN 5.31 workstation. Briefly, clear supernatant was loaded onto a mono-Q anion-exchange chromatography HP column (5 mL, GE Healthcare) that had previously been equilibrated with 50 mm Tris, 100 mm NaCl, 10% glycerol pH 8. The protein was then eluted with an increasing NaCl gradient (0 to 1 M) for 100 min at a rate of 1 mL/min. Eluted fractions containing the protein of interest were pooled and concentrated using Millipore Vivaspin20 10 kDa (Sartorius). These were then injected into a superdex 75 (16/60) gel-filtration column (GE Healthcare) that had been equilibrated with 50 mM Tris, 150 mM NaCl, 10% glycerol pH 8.5. Fractions were assessed with SDS-PAGE to determine purity, and the protein concentration was calculated spectroscopically using the extinction coefficient at 280 nm.

### Purification and Refolding of Recombinant β-Etherase

Cell cultures were pelleted through centrifugation. Supernatant was discarded, and pellets were suspended in 5 mL per 100 mL of starting culture 20 mM (4-(2-hydroxyethyl)-1-piperazineethanesulfonic acid) (HEPES) pH 8, before sonicated on ice (70 V, 4 s on, 7 s off for a total of 4 min on). Centrifugation at 10 000 xg was again used to pellet cell debris and inclusion bodies. The pellet was washed with 20 mM HEPES, 2 M Urea, 0.5 M NaCl, 2% TritonTM X-100, pH 8, using the same volume as before, and sonicated and centrifuged as before. The resultant pellet was then resuspended in 20 mM HEPES, 0.5 M NaCl, 5 mM imidazole, 6 M guanidine hydrochloride, 1 mM dithiothreitol (DTT) pH 8, using 10 mL per 100 mL of original cell culture, to solubilise inclusion bodies. After pelleting through centrifugation for a final time, the supernatant was applied to a HisTrap column equilibrated with 20 mM HEPES, 0.5 M NaCl, 5 mM imidazole, 6 M guanidine hydrochloride, 1 mM DTT pH 8. The equilibration buffer was then used to wash the column for a total of 5 CV followed by the same volume of 20 mM HEPES, 0.5 M NaCl, 20 mM imidazole, 6 M urea, 1 mM DTT pH 8. A linear gradient from the final wash buffer to 20 mM HEPES, 0.5 M NaCl, 20 mM imidazole, 0.1 mM CuSO₄, 1 mM DTT pH 8 was then used to refold the tagged protein on the column. This was applied over 30 mL using a flow rate of 0.5 ml/min. To elute refolded protein another linear gradient was applied over 20 mL, starting with 20 mM HEPES, 0.3 M MgCl₂, 20 mM imidazole, 1 mM DTT, pH 8 and ending with the same buffer with the addition of 500 mM imidazole and 10% glycerol. Apart from when otherwise mentioned, the flow rate was kept at 1 mL/ min when using a 1 mL capacity column and 3 mL/min when using a 5 mL capacity column. Fractions of 1.5 mL were collected throughout the elution step, and UV absorbance was used to determine protein content. Fractions with high protein contents were visualised using SDS-polyacrylamide gel electrophoresis (SDS-PAGE) and the presence of the recombinant protein confirmed through western blot analysis. Protein activity was confirmed through the measurement of 4MU from the GGβ4MU assay after removal of imidazole and DTT using Zeba™ Spin Desalting Columns, 7K MWCO (ThermoFisher) or Slide-A-Lyzer™ Dialysis Cassettes 10 K MWCO (ThermoFisher).

### Fluorescence Assay for β-Etherase

Enzyme activity was measured in 1 mL reaction containing 10 µL 4MU/GGβ4MU (synthetic fluorescent substrate 10 mM) and appropriate concentration of pure protein in 50 mM Tris-HCI, 100 mM NaCl, pH 8.5, 5 mM CuSO₄. The reaction was incubated at 30° C. for 1 h. Formation of 4-methylumbelliferone (4MU) was monitored using an RF-1500 fluorometric analyzer. After 0 h and 1 h of incubation 100 µL of the reaction mixture was taken and added to 50 µL of 100 mM glycine-NaOH buffer (pH 10.0). One unit of the enzyme was defined as the amount that released 1 nmol of 4 MU/h from the substrate. Five replicate were taken for each sample, and control reactions of boiled enzyme and wheat straw treated with buffer only were also performed.

### Enzyme Properties

The effect of pH and temperature on enzyme activity was investigated by varying the pH of the reaction mixtures using 50 mM Tris-HCI buffer from pH 7.0 to 9.5, 50 mM glycine-NaOH buffer at pH range 9.0 to 10.5 and 50 mM Na₂HPO₄-NaOH buffer at pH range 10.5 to 12. The optimum temperature of enzyme activity was determined at various temperatures ranging from 20° C. to 70° C. Assays were performed as described in the previous section.

### Phenol Oxidase Assay

Specificity was investigated by incubating 1 mM of each substrate of interest with the enzyme in 100 µL Tris pH 8.5 buffer at room temperature. Activity was determined by monitoring the change in Ultraviolet-Visible absorbance spectra (220 - 750 nm) of aliquots using a NanoDrop 8000 Microvolume UV-Vis spectrophotometer (Thermo Scientific). Scans were performed at regular intervals over 2 h.

### Extraction of Tricin

Wheat straw was ground to <1 mm using a cyclone mill (Retsch) and washed several times with 50 mM Tris pH 8 to remove residual surface sugars. In 1 mL reactions, 100 mg of washed wheat straw was incubated with an appropriate concentration of pure enzyme in 50 mM Tris buffer at pH 8 with 5 mM CuSO₄. Reactions were incubated overnight at 30° C. with shaking. Control reactions were performed using wheat straw incubated with boiled β-etherase or with buffer only. Tricin was extracted based upon Karambelkar.⁷⁰ Briefly, 1 mL of ethyl acetate was added to 100 µL of the reaction supernatant. This was homogenized before being centrifuged for 5 min at 13,000 rpm. The ethyl acetate layer was transferred into new tubes and evaporated using a centrifugal evaporator at 55° C. before being resuspended in 100 µL 50% H₂O, 50% acetonitrile. This was analyzed with a Waters 2996 photodiode array detector Separations Module HPLC system, column used was C18-5 µM preparative column (4.6 × 250 mm, Waters, X-Bridge, Made in Ireland). The mobile phase was 0.1% acetic acid in water (A), and methanol (B) and a linear gradient was used; 95% A (5 min), 70% A (25 min), 0% A (30 min), 95% A (5 min), the flow rate was 1.0 mL/min. After identification through comparisons with authentic standards, based on retention time and UV spectrum, peaks were manually collected and the mass confirmed with mass spectroscopy.

### β-Etherase Boosting Saccharification with Cellulase Enzymes

For saccharification reactions, biomass pretreated with β-etherase was incubated with 1.2 µg/mL enzyme cocktail (4:1 Celluclast: novo 188 (Novozymes)) in 50 mM sodium acetate at pH 4.5 and incubated overnight at 37-40° C. with shaking. This was performed alongside a control reaction with buffer only. Solids were removed by centrifugation, and residual protein was precipitated with 80% ethanol. The supernatant, containing mono- and oligosaccharides, was dried with a centrifugal evaporator before samples were resuspended in ultra-pure water and filtered through a 0.2 µm polytetrafluoroethylene (PTFE) filter. Five replicates from each sample were investigated, and carbohydrate composition was analyzed by high-performance anion-exchange chromatography (HPAEC).

### High-Performance Anion-Exchange Chromatography (HPAEC)

High-performance anion-exchange chromatography was used to analyze monosaccharide release after saccharification. Briefly, 5 µL of samples or standards were injected on a CarboPac PA20 3 × 150 mm analytical column via a CarboPac PA20 3×0 mm guard column using Chromeleon 6.8 Chromatography Data Systems software (Dionex). Sugars were separated at a flow rate of 0.4-0.5 mL min⁻¹ at a temperature of 25° C. as follows: after equilibration of the column with 100% H₂O, samples were separated in a linear gradient of 100% H₂O to 99%-1% of H₂O-0.2 M NaOH for 5 min, then constant for 10 min, followed by a linear gradient to 47.5%-22.5%-30% of H₂O-0.2 M NaOH-0.5 M NaOAc/0.1 M NaOH in 7 min and then kept constant for 15 min. After washing the column with 0.2 M NaOH for 8 min it was re-equilibrated with 100% H₂O for 10 min before the injection of the next sample. Carbohydrates were detected by ICS-3000 PAD system with an electrochemical gold electrode, identified by comparison with retention times of external standards (arabinose, fucose, galactose, glucose, glucuronic acid, mannose, rhamnose, and xylose) and quantified through the integration of these known standards.

### Lignin Isolation

Enzyme lignins, representing essentially all of the lignin in the sample, were prepared following ball-milling of the cell wall isolate as previously described.(75-77, 78)

### NMR Analysis

2D NMR of enzyme lignins (EL) in 4:1 v/v DMSO-d₆:pyridine-d₅ were acquired on a Bruker Biospin (Billerica, MA) Avance 700 MHz spectrometer equipped with a 5-mm QCI ¹H/³¹P/¹³ C/¹⁵ N QCI cryoprobe with inverse geometry (proton coils closest to the sample), as described previously.(76,77) Volume-integration of contours in HSQC plots used TopSpin 4.07 (Mac version) software, and no correction factors were used. The data represent volume-integrals only, and data are presented on an S + G + H = 100% basis (FIG. 17); pCA, and tricin T units are always terminal and are, therefore, likely overestimated.(77) Data assignments here were made by comparison with published data from other samples from our lab, including in the various tricin-related papers.(71-74, 79, 80)

### Statistical Analysis

Where mentioned two tail ANOVAs were performed using R core package “stats”.(83)

### Example 1 Isolation of Parascedosporium Putredinis NO1

We inoculated liquid cultures containing wheat straw as the sole carbon source with samples of wheat straw-enriched compost and tracked the dynamics of the resulting microbial community using targeted amplicon sequencing during cultivation. Sequencing of 16S ribosomal RNA genes generated over three million reads from the prokaryotic community over the whole time course, which clustered together to form 25,304 operational taxonomic units (OTUs) (FIG. 1a). The most abundant bacterial phyla identified were the gram-negative Bacteroidetes, Verrucomicrobia and Proteobacteria, respectively, representing an average of 31%, 19.8%, and 15.5% of the total reads across the time course. Analysis of the eukaryotic community by sequencing the Internal Transcribed Spacer (ITS) region predominantly yielded reads that had no match within the UNITE fungal rDNA sequence database.23,24 In total, 96.5% of generated OTUs were not recognized as fungal and instead showed the closest homologies to protozoa. Among the fungi, we noted distinct changes in the composition of the community with time. In particular, a fungus (designated strain NO1) identified as Parascedosporium putredinis an Ascomycete in the Microascaceae family, showed increased abundance after 4 weeks of incubation (FIG. 1b). This fungus was readily isolated from shake flasks by culturing on both nutrient agar and potato dextrose agar and dominated the eukaryotic community in the shake flasks after four weeks of incubation, representing 84% of the identifiable fungal reads at 8 weeks, a time point by which, we hypothesize, the majority of easily accessible carbon from wheat straw has been depleted.²⁵ Interestingly, this fungus could be selectively cultivated when agar plates contained kraft lignin as the sole carbon source.

### Example 2 Omics Analysis of Wheat Straw Degradation by P. Putredinis NO1

We confirmed that P. putredinis NO1 could grow on wheat straw as a sole carbon source and optimized the composition of growth media for cellulase and xylanase production using a central composite design (FIG. 6). The deconstruction of wheat straw by P. putredinis NO1 over 28 days was subsequently tracked by measuring mass loss and carbohydrate-active enzyme (CAZy) activity (FIG. 7). From this study, we identified the second, fourth and tenth day of incubation on wheat straw as distinct time points to harvest RNA for sequencing on an Illumina platform. These incubation times were chosen as together they represent the first detection of lignocellulolytic activity (day 2), the peak of enzyme activities (day 4) and the subsequent reduction of lignocellulolytic activity - a point at which the easily accessible sugars in the wheat straw had been utilized. In total, 5,586 unique contiguous DNA sequences (contigs) were assembled from the 339,854,704 reads generated, and differential gene analysis identified 2,189 contigs that were upregulated at high confidence and fold change (P<0.001, FC >10) when P. putredinis NO1 was grown on wheat straw compared to growth on glucose. These highly upregulated genes included those coding for 102 putative CAZy proteins; comprising 47 glycoside hydrolases (GH), 41 auxiliary activities (AA), ten carbohydrate esterases (CE) and a polysaccharide lyase (PL). The majority of CAZy family proteins were upregulated after four days of growth (FIG. 2), in agreement with the peak of the observed enzymatic activities in P. putredinis NO1 culture supernatants.

As the macromolecular structure of lignocellulose prohibits intracellular degradation, many enzymes for its deconstruction must be secreted. We therefore performed LC-MS/MS analysis on protein samples collected directly from the culture supernatant, and separately from those bound to insoluble components of the culture using a biotin-labelling method designed to enrich for proteins tightly bound to the residual biomass.²⁶ We identified 3,671 proteins across all samples, including 1,037 proteins present in only wheat straw conditions (FIG. 8a). Within the resultant protein library, 275 sequences contained a recognizable CAZy domain. These accounted for 25.7% (194 proteins) of the molar percentage of the supernatant samples and 14.1% (174) of the biotin-labelled samples after four days of growth on wheat straw, compared to 13.3% (97) of the supernatant and 2% (56) of the biotin labelled samples from glucose-grown cultures (FIG. 8b).

The most abundant CAZy protein family, accounting for 3.7% and 3.6% of the respective supernatant and biotin-labelled fractions on the fourth day, were GH6s, which may be endoglucanases or processive cellobiohydrolases. These, along with GH7s, often constitute the major cellulases in filamentous fungi.²⁷ The GH6 family, is represented by four distinct proteins within the proteome, included the most abundant single protein - c7229_g3_i1_1, a putative cellobiohydrolase with an 85.89% sequence identity to a cellulase (XP_016646396.1) from Scedosporium apiospermum. Other abundant GHs likely active on cellulose include GH7 (typically cellobiohydrolases or endoglucanases), GH5 and GH45 (often endoglucanases) and GH1 and 3 (typically glucosidases).²⁸

Efficient lignocellulose deconstruction demands a combination of cellulolytic and hemicellulolytic enzymes that work cooperatively. Enzymes related to the depolymerization of arabinoxylan (major hemicellulose of wheat straw), were well represented within the exoproteome. Nine proteins were identified with homology to endo β-1-4-xylanases (GH10 and GH11), which hydrolyse the arabinoxylan backbone, and five proteins were identified as putative β-1,4-xylosidases that act on the resultant fragments to produce xylose monomers (GH3, GH31, GH43_1, GH43_11, GH43_36). Also of note were the GH43 subfamilies GH43_1, GH43_21, GH43_22, GH43_26 and GH43_36 that were abundant within the secretome, including putative β-D-xylosidases, α-L-arabinofuranosidase, and β-1,3-galactosidase activities. Fifteen GH43 subfamily members were identified, with nine proteins showing closest homology to known arabinofuranosidases.

Three proteins, belonging to the CE1 family, showed significant sequence homology to feruloyl esterases. Ferulic acid is esterified to the arabinose side chain of arabinoxylans, and through the formation of diferulate bridges and ester-ether linkages allows the respective formation of covalent interactions between arabinoxylan chains and lignin. Feruloyl esterases, therefore, are thought to aid the solubilization of plant cell wall polysaccharides by the hydrolysis of the ester link that exists between ferulic acid residues and arabinose, thereby disrupting the crosslinking of cell wall components.²⁹ Putative acetyl xylan esterases (3 in CAZy family CE1 and 3 in CE5) were also observed and are known to facilitate the degradation of xylan through the removal of acetyl substitutions.³⁰

The CAZy auxiliary activity (AA) class is classified as containing enzymes that act in conjunction with carbohydrate-active enzymes through redox activities. Interestingly, 69 putative proteins from the AA class were detected in the exosecretome, more than many lignocellulose-degrading fungi contain in their total genome,³¹ suggesting an important role for the oxidative degradation of lignocellulose in P. putredinis NO1. The AA9 family, which along with the AA10, AA11, AA13, AA14 and AA15 families constitute the lytic polysaccharide monooxygenases (LPMOs) - a class of copper metalloenzymes that catalyse the oxidative cleavage of glycosidic bonds in multiple polysaccharide substrates including chitin, cellulose, and xylan,^{32.33} were highly represented within the exosecretome. In total, we identified nineteen putative LPMOs (16 AA9s; 2 AA11s; 1 AA13), fifteen of which were upregulated tenfold or more between glucose and wheat straw conditions. Fittingly, 16 AA3s (glucose-methanol-choline (GMC) oxidoreductase) and 9 AA7s (glucooligosaccharide oxidase), which have been shown to facilitate the activity of the LPMOs through electron shuttling,^{34,35} were also present within wheat straw cultures.

Five putative multicopper oxidase proteins were also observed - two from the AA1_3 subfamily (Laccase-like multicopper oxidase) and one from the AA1_2 subfamily (Ferroxidase). Laccase-like multicopper oxidases are of unknown function but have been implicated in lignin degradation, as well as other diverse functions (iron homeostasis, offense/defence),³⁶ whereas ferroxidases have been reported to be involved in lignocellulose degradation in Ascomycetes, in which they generate hydroxyl radicals via the Fenton reaction.³⁷ Established lignin depolymerizing enzymes associated with the white-rot fungal decay of lignin, including laccases from the AA1_1 subfamily or peroxidases from the AA2 family, were not present within the libraries, perhaps not surprising given the P. putredinis NO1 sits within the Ascomycota phylum, and as such is closer in relation to the soft-rots.

Despite the apparent lack of known ligninases in P. putredinis NO1, a putative AA6 (1,4-benzoquinone reductase) associated with the intracellular biodegradation of aromatic compounds was present within the supernatant and may have a role in the metabolism of lignin breakdown products.^{31,38}

Of key interest to us was the potential of P. putredinis NO1 to produce novel lignocellulolytic activities, particularly those able to boost lignocellulose deconstruction via the modification and solubilization of lignin. An unknown protein, c2092, identified in the exosecretome was subsequently found to have β-etherase activity and no CAZy identification.

### Example 3 A New β-Etherase

The β-ether motif with its characteristic β—O—4 inter-unit linkage is the most abundant in lignin, estimated at representing over 50% of the total inter-unit linkages.³⁹ Enzymes employing β-ether cleavage mechanisms can deconstruct synthetic and extracted lignin;^{40,41,42} these bacterial etherases that have been characterized to date, however, are intracellular proteins, and are glutathione- or NAD⁺- dependent, suggesting that in nature they are not directly involved in the breakdown of the lignin macromolecule, but rather its smaller, membrane-transportable oligomers. An extracellular fungal protein displaying β-etherase activity was previously purified from the supernatant of the Chaetomium sp. 2BW- 1, although its identity remains unknown.⁴³

Using a synthetic lignin model compound GGβ4MU (7-[2-hydroxy-2-(4-hydroxy-3-methoxyphenyl)-1-(hydroxymethyl)ethoxy]-4-methyl-2/7-1-benzopyran-2-one) containing a β-methylumbelliferyl ether, guaiacylglycerol-β-(4-methylumbelliferyl) ether (FIG. 9),⁴⁴ that when cleaved yields the fluorogenic product 4-methylumbelliferone (4MU), we detected β-etherase activity within the culture supernatant of P. putredinis NO1. This activity was present when P. putredinis NO1 was grown on wheat straw but not on glucose, suggesting a possible role in lignocellulose degradation, and appeared to be independent of cofactors such as glutathione or NAD⁺. Given its presence in the secretome and its apparent cofactor independence, we hypothesized that this putative ligninase was unlikely to share significant sequence homology to the previously described intracellular β-etherases from sphingomonads, and indeed no proteins with similarity to these enzymes were detected. We, therefore, subjected the culture supernatant of P. putredinis NO1 grown on wheat straw to a series of protein fractionation techniques, enriching at each step for β-etherases activity.

The putative β-etherase was initially purified by ammonium sulfate precipitation of the proteins in the culture supernatant to decrease sample pigmentation and reduce protein-protein interactions. This treatment facilitated further purification by size-exclusion and anion-exchange chromatography. Using shotgun proteomics, we identified c2092, a 44.5 kDa protein present in the purified fraction that contained a predicted signal peptide. Analysis of the transcriptomic and proteomic data revealed this protein was strongly upregulated in the presence of wheat straw and present in both the supernatant and biotin-labelled proteomic libraries throughout the growth of P. putredinis NO1 on wheat straw (FIG. 10). Using profile Hidden Markov models constructed by HMMER3 on using the pFAM database,⁴⁵ we saw homology to a common central tyrosinase domain (PF00264; Evalue = 7.1e-49) with a characteristic binuclear type-3 copper-binding site consisting of six histidine residues located in a four-helical bundle coordinating the binding of two copper ions⁴⁶ (FIG. 11). Fungal tyrosinases are associated with pigmentation and browning; specifically, through melanin production, whereby they catalyse the introduction of a hydroxyl group at the ortho-position of a para-substituted monophenols and the subsequent oxidation to the corresponding o-quinone.⁴⁷ However, c2092 lacks both the C- and N-terminal domains that tyrosinases typically contain and instead shows higher homology (170/370 identity (46%)) to a catechol oxidase (AoCO4) from Aspergillus oryzae.⁴⁸ Catechol oxidases differ from tyrosinases due to a lack of mono-oxygenase activity.⁴⁹ Examination of the proteomics library resulted in the identification of seven sequences with significant similarities to c2092 (Table 1), all predicted to be extracellular and soluble, and five upregulated in the presence of wheat straw (FIG. 12). Searches within the NCBI non-redundant database further revealed the presence of proteins of similar sequence (>50% sequence identity) distributed throughout fungal genomes of the Sordariomycetes class of Ascomycetes (Table 2).

### Example 4 Experimental Confirmation of β-Etherase Activity

To determine if c2092 was responsible for the observed β-etherase activity, we heterologously expressed the codon-optimized sequence in Escherichia coli. The recombinant protein was purified (Table 3), and the β-etherase activity of the protein was confirmed by determining the level of fluorescence released after incubation with GGβ4MU (FIG. 13a). The pH and temperature dependency of the enzyme were investigated, revealing maximum activity at pH 10 and 60° C. (FIGS. 13b-c). Whereas the mushroom tyrosinase (Agaricus bisporus) has been reported to have promiscuous β-etherase activity on small synthetic compounds, no significant activity has been reported against macromolecular lignin.⁵⁰ The β-etherase from P. putredinis NO1 did not display activity against L-tyrosine and L-DOPA, as is characteristic of tyrosinases (FIG. 14).⁵¹ We subsequently assayed for potential oxidase activity against a range of phenolic substrates, including di-phenolics, known to be catechol oxidase substrates,⁴⁹ and observed no similarities to catechol oxidase in terms substrate preferences (FIG. 15, Table 4). Interestingly, the etherase showed activity with the substrates: 4-hydroxybenzoic acid, vanillic acid, and quercetin, all known to be tyrosinase inhibitors.⁵²

### Example 5 Release of Tricin and Lignin Units from Wheat Straw

Tricin has recently been described as a subunit in the lignin of monocot species, incorporated through a 4—O—β linkage.¹¹ As wheat straw contains relatively high concentrations of tricin compared to other agriculturally relevant feedstocks,⁸ we assessed the ability of the β-etherase to release tricin from wheat straw. The β-etherase was incubated with wheat straw for sixteen hours under physiological conditions (pH 8.5 and 30° C.). Reaction products were monitored by High-Performance Liquid-Chromatography (HPLC), and a peak corresponding to tricin was identified by reference to an authentic standard and confirmed by mass spectrometry. Under the growth conditions used for P. putredinis NO1, a significantly higher concentration of tricin was present in the reaction supernatant of wheat straw with the β-etherase compared to incubations with buffer alone (ANOVA, F(2,12)=44.67, p<0.05) (FIG. 4a). We were also able to detect the presence of p-coumaric acid, vanillin, and p-hydroxybenzaldehyde in the reaction supernatant through comparisons with authentic standards and mass spectrometry; however, unlike tricin, these compounds were not enriched under the β-etherase-treated reaction conditions (FIG. 16c) and presumably are produced as a result of simple ester cleavage.

NMR (FIG. 17) of the enzyme lignins (EL) isolated (following crude polysaccharidase treatment to saccharify most of the polysaccharides),(75) and the product generated from it by a non-optimized treatment with our enzyme showed little change to the actual lignin profile but a strong decrease in the tricin level. Thus, even though integration of correlation contours in the spectra resulting from such 2D-HSQC (heteronuclear single-quantum coherence) experiments does not provide reliable quantification, their relative values are considered to be valid.(76,77) Analysis showed that the relative tricin ether level in the lignin dropped from nearly 12% in the control to about 8.5% after the treatment. We were initially disappointed that we couldn’t detect similar reductions in levels of the β-ether units A (FIG. 17), but caution that these are ‘quantified’ on an A+B+C=100% basis and it is easy to speculate on how the levels might not significantly change even with some (presumably low-level) β-ether cleavage. In spectra from the whole cell wall component (and not just the isolated lignin, not shown), the trends were similar and the T6 and T8 contours were particularly weak in the treated sample whereas the T2′/6′ peak was relatively strong; we have noted this occurrence before in rapidly relaxing samples, and do not fully understand its origin; regardless, the relative tricin level in the treated material was again lower than in the control and obviously consistent with the measured release of tricin noted above.

We further tested the activity of the β-etherase on alternative feedstocks, including sugarcane bagasse and rice straw. A smaller amount of tricin was released from sugarcane bagasse compared to wheat straw; however, in contrast to assays with wheat straw, p-coumaric acid was significantly enriched (ANOVA, F(2,12)=44.67, p<0.05) (FIG. 4b, FIG. 16). Rice straw showed little difference in product release, with relatively low concentrations of tricin and p-coumaric acid released during the incubation (FIG. 16).

As mushroom tyrosinase has been reported to cleave β-ether linkages promiscuously,⁵⁰ we tested its β-etherase activity on these lignocellulosic substrates under equivalent conditions. We observed less tricin, p-coumaric acid, and p-hydroxybenzaldehyde production in the reaction mixtures containing mushroom tyrosinase compared to the P. putredinis NO1 β-etherase treatments. Tricin is a known tyrosinase inhibitor that binds non-competitively to the hydrophobic pocket of the protein,⁵³ and p-coumaric acid has been characterized as having a mixed-type inhibition effect.⁵⁴ This inhibition, through the non-reversible binding of the reaction products, could go some way to explaining why mushroom tyrosinase displays little activity towards the lignin macromolecule.

### Example 6 β-Etherase Pretreatment Boosts Saccharification

The recalcitrance of lignocellulose to degradation requires that feedstocks are pretreated in order to disrupt lignin, before efficient saccharification can be achieved using current commercial enzymatic cocktails. These pretreatments are typically physico-chemical, using a combination of heat and pressure with acid, alkali or organic solvents. As these industrial processes are energy-intensive and environmentally damaging, the use of biological treatments, performed under relatively mild conditions, are desirable. To investigate if the application of the β-etherase would improve saccharification rates, we treated sugarcane bagasse, wheat straw, and rice straw with β-etherase for sixteen hours before the addition of commercial cellulases. Sugarcane bagasse demonstrated a major improvement in digestibility after pretreatment with β-etherase resulting in a significant increase in glucose, xylose, and arabinose compared to the untreated control (2-fold, 5-fold and 23-fold, respectively) after saccharification (FIGS. 5a-b). Wheat straw treated with β-etherase also showed an improvement in glucose release (ANOVA, F(2,12)= 4.47, p<0.05), albeit at a more modest level with a 1.2-fold increase. Interestingly, no improvement in saccharification was observed with rice straw, which may reflect the lower lignin content of rice straw compared to wheat straw and sugarcane.⁵⁵ This suggests that although the β-etherase can modify the plant cell wall structure and enhance digestibility, differences in lignocellulose organization and lignin content between feedstocks may determine the extent to which this occurs.

### Example 7: Enzyme Homology and Identification

P. putredinis NO1 is able to dominate cultures in the latter stages of wheat straw degradation in a mixed microbial community, in liquid culture, when easily accessible polysaccharides have been exhausted. Using a combination of omics approaches, we have identified a diverse range of potentially industrially relevant carbohydrate-active enzymes, including a large number of enzymes associated with the oxidative attack on lignocellulose. In particular, we have identified a new extracellular β-etherase that is preferentially expressed in the presence of wheat straw and demonstrated that this enzyme can boost enzymatic hydrolysis by cellulases as well as selectively release the pharmaceutically relevant flavonoid tricin from monocot lignin. The cleavage of β-ether bonds most likely aids the breakdown of lignocellulose in natural environments. We contend that this ability to deconstruct and modify lignin is important for P. putredinis NO1 to be able to out-compete other microbial species during the latter stage of plant biomass degradation. Preferential removal of tricin subunits has been described by the white-rot fungi, Pleurotus eryngii, during the selective delignification of wheat straw and has been proposed to be key to lignocellulose degradation, although the enzyme activity that facilitated tricin release was not identified.⁵⁶ When the publicly available genome of P. eryngii was examined for the presence of proteins with homology to the β-etherase from P. putredinis NO1 no significant hits were detected. As the protein described as being responsible for β-etherase activity from Chaetomium sp. 2BW-1 was not identified to sequence level, it is unclear whether it shares homology to the enzyme described here; however, the proteins appear to be distinct as the reported sizes differ by 20 kDa.⁴³ Taken together, these observations suggest that multiple, structurally dissimilar, enzymes in the natural environment may mediate ether linkage disruption in lignocellulose-degrading microbes. To the best of our knowledge, this is the first identification and characterization of an extracellular β-etherase that has no cofactor requirement for activity capable of selectively releasing tricin from lignin and could have potential biotechnological applications.

### REFERENCES

8. Lan W, et al. Tricin-lignins: occurrence and quantitation of tricin in relation to phylogeny. 88, 1046-1057 (2016).
11. Li M, pu Y, Yoo CG, RagauskasA. The occurrence of tricin and its derivatives in plants. Green Chem 18, (2016).
23. Kõljalg U, et al. UNITE: a database providing web-based methods for the molecular identification of ectomycorrhizal fungi. 166, 1063-1068 (2005).
24. Abarenkov K, et al. The UNITE database for molecular identification of fungi - recent updates and future perspectives. 186, 281-285 (2010).
25. Alessi AM, et al. Defining functional diversity for lignocellulose degradation in a microbial community using multi-omics studies. Biotechnol Biofuels 11, 166 (2018).
26. Alessi AM, et al. Revealing the insoluble metasecretome of lignocellulose-degrading microbial communities. Scientific reports 7, 2356 (2017).
27. Jun H, Guangye H, Daiwen C. Insights into enzyme secretion by filamentous fungi: Comparative proteome analysis of Trichoderma reesei grown on different carbon sources. Journal of Proteomics 89, 191-201 (2013).
28. Glass NL, Schmoll M, Cate JHD, Coradetti S. Plant cell wall deconstruction by ascomycete fungi. 67, 477-498 (2013).
29. de Oliveira DM, et al. Ferulic acid: a key component in grass lignocellulose recalcitrance to hydrolysis. Plant biotechnology journal 13, 1224-1232 (2015).
30. Zhang J, Siika-Aho M, Tenkanen M, Viikari L. The role of acetyl xylan esterase in the solubilization of xylan and enzymatic hydrolysis of wheat straw and giant reed. Biotechnol Biofuels 4, 60 (2011).
31. Levasseur A, Drula E, Lombard V, Coutinho PM, Henrissat B. Expansion of the enzymatic repertoire of the CAZy database to integrate auxiliary redox enzymes. Biotechnol Biofuels 6, 41 (2013).
32. Vaaje-Kolstad G, et al. An oxidative enzyme boosting the enzymatic conversion of recalcitrant polysaccharides. Science 330, 219-222 (2010).
33. Quinlan RJ, et al. Insights into the oxidative degradation of cellulose by a copper metalloenzyme that exploits biomass components. 108, 15079-15084 (2011).
34. Laurent C, Breslmayr E, Tunega D, Ludwig R, Oostenbrink C. Interaction between cellobiose dehydrogenase and lytic polysaccharide monooxygenase. Biochemistry 58, 1226-1235 (2019).
35. Tan T-C, et al. Structural basis for cellobiose dehydrogenase action during oxidative cellulose degradation. Nature Communications 6, 7542 (2015).
36. Levasseur A, et al. Exploring laccase-like multicopper oxidase genes from the ascomycete Trichoderma reesei: a functional, phylogenetic and evolutionary study. BMC Biochemistry 11, (2010).
37. Kersten P, Cullen D. Extracellular oxidative systems of the lignin-degrading basidiomycete Phanerochaete chrysosporium. Fungal Genetics and Biology 44, 77-87 (2007).
38. Daly P, et al. Expression of Aspergillus niger CAZymes is determined by compositional changes in wheat straw generated by hydrothermal or ionic liquid pretreatments. Biotechnol Biofuels 10, 35 (2017).
39. Schutyser W, Renders T, Van den Bosch S, Koelewijn SF, Beckham GT, Sels BF. Chemicals from lignin: an interplay of lignocellulose fractionation, depolymerisation, and upgrading. Chemical Society reviews 47, 852-908 (2018).
40. Gall DL, et al. In vitro enzymatic depolymerization of lignin with release of syringyl, guaiacyl, and tricin units. Applied and environmental microbiology 84, (2018).
41. Kontur WS, et al. A heterodimeric glutathione S-transferase that stereospecifically breaks lignin’s β(R)-aryl ether bond reveals the diversity of bacterial β-etherases. The Journal of biological chemistry 294, 1877-1890 (2019).
42. Marinovic M, et al. Selective cleavage of lignin β—O—4 aryl ether bond by β-etherase of the white-rot fungus Dichomitus squalens. ACS Sustain Chem Eng 6, 2878-2882 (2018).
43. Otsuka Y, Sonoki T, Ikeda S, Kajita S, Nakamura M, Katayama Y. Detection and characterization of a novel extracellular fungal enzyme that catalyzes the specific and hydrolytic cleavage of lignin guaiacylglycerol β-aryl ether linkages. 270, 2353-2362 (2003).
44. Weinstein DAG, M.H. Synthesis of guaiacylglycol and glycerol-β—O—(β-methylumbelliferyl) ethers: lignin model substrates for the possible fluorometric assay of β-etherases. Holzforschung 33, 134-135 (1979).
45. Finn RD, et al. The Pfam protein families database. Nucleic Acids Research 38, D211-D222 (2010).
46. Kanteev M, Goldfeder M, Fishman A. Structure-function correlations in tyrosinases. Protein Science 24, 1360-1369 (2015).
47. Halaouli S, Asther M, Sigoillot JC, Hamdi M, Lomascolo A. Fungal tyrosinases: new prospects in molecular characteristics, bioengineering and biotechnological applications. Journal of Applied Microbiology 100, 219-232 (2006).
48. Hakulinen N, Gasparetti C, Kaljunen H, Kruus K, Rouvinen J. The crystal structure of an extracellular catechol oxidase from the ascomycete fungus Aspergillus oryzae. Journal of biological inorganic chemistry : JBIC : a publication of the Society of Biological Inorganic Chemistry 18, 917-929 (2013).
49. Gasparetti C, Faccio G, Arvas M, Buchert J, Saloheimo M, Kruus K. Discovery of a new tyrosinase-like enzyme family lacking a C-terminally processed domain: production and characterization of an Aspergillus oryzae catechol oxidase. Applied Microbiology and Biotechnology 86, 213-226 (2010).
50. Min K, et al. Perspectives for biocatalytic lignin utilization: cleaving 4-O-5 and Cα—Cβ bonds in dimeric lignin model compounds catalyzed by a promiscuous activity of tyrosinase. Biotechnol Biofuels 10, 212 (2017).
51. Yang Z, Robb DA. Comparison of tyrosinase activity and stability in aqueous and nearly nonaqueous environments. Enzyme and Microbial Technology 15, 1030-1036 (1993).
52. Zolghadri S, et al. A comprehensive review on tyrosinase inhibitors. J Enzyme Inhib Med Chem 34, 279-309 (2019).
53. Mu Y, Li L, Hu S-Q. Molecular inhibitory mechanism of tricin on tyrosinase. Spectrochimica Acta Part A: Molecular and Biomolecular Spectroscopy 107, 235-240 (2013).
54. Lim JY, Ishiguro K, Kubo I. Tyrosinase inhibitory p-coumaric acid from ginseng leaves. PhytotherRes 13, 371-375 (1999).
55. Van Soest PJ. Rice straw, the role of silica and treatments to improve quality. Animal Feed Science and Technology 130, 137-171 (2006).
56. van Erven G, Nayan N, Sonnenberg ASM, Hendriks WH, Cone JW, Kabel MA. Mechanistic insight in the selective delignification of wheat straw by three white-rot fungal species through quantitative ¹³C-IS py-GC-MS and whole cell wall HSQC NMR. Biotechnol Biofuels 11, 262 (2018).
57. Caporaso JG, et al. QIIME allows analysis of high-throughput community sequencing data. Nature methods 7, 335-336 (2010).
58. DeSantis TZ, et al. Greengenes, a chimera-checked 16S rRNA gene database and workbench compatible with ARB. Applied and environmental microbiology 72, 5069-5072 (2006).
59. McDonald D, et al. An improved Greengenes taxonomy with explicit ranks for ecological and evolutionary analyses of bacteria and archaea. ISME J 6, 610-618 (2012).
60. Abarenkov K, et al. The UNITE database for molecular identification of fungi-recent updates and future perspectives. The New phytologist 186, 281-285 (2010).
61. Bezerra MA, Santelli RE, Oliveira EP, Villar LS, Escaleira LA. Response surface methodology (RSM) as a tool for optimization in analytical chemistry. Talanta 76, 965-977 (2008).
62. Lever M. Colorimetric and fluorometric carbohydrate determination with p-hydroxybenzoic acid hydrazide. Biochemical Medicine 7, 274-281 (1973).
63. Finn RD, Clements J, Eddy SR. HMMER web server: interactive sequence similarity searching. Nucleic acids research 39, W29-W37 (2011).
64. Camacho C, et al. BLAST+: architecture and applications. BMC bioinformatics 10, 421 (2009).
65. Altschul SF, Gish W, Miller W, Myers EW, Lipman DJ. Basic local alignment search tool. Journal of molecular biology 215, 403-410 (1990).
66. Emanuelsson O, Brunak S, von Heijne G, Nielsen H. Locating proteins in the cell using TargetP, SignalP and related tools. Nature protocols 2, 953-971 (2007).
67. Petersen TN, Brunak S, von Heijne G, Nielsen H. SignalP 4.0: discriminating signal peptides from transmembrane regions. Nature methods 8, 785-786 (2011).
68. Ishihama Y, et al. Exponentially modified protein abundance index (emPAI) for estimation of absolute protein amount in proteomics by the number of sequenced peptides per protein. Molecular & cellular proteomics : MCP4, 1265-1272 (2005).
69. H. Pages PA, R. Gentleman and S. DebRoy. BioStrings: Efficient manipulation of biological strings. (ed^(eds). R package version 2.52.0. edn (2018).
70. Karambelkar P, Jadhav, V.M. , Kadam, V. Isolation and characterization of flavonoid tricin from sugarcane sludge. Indo American Journal of Pharmaceutical Research 4, 7 (2014).
71. J. C. del Rio et al., Structural characterization of wheat straw lignin as revealed by analytical pyrolysis, 2D-NMR, and reductive cleavage methods. Journal of Agricultural and Food Chemistry 60, 5922-5935 (2012).
72. W. Lan et al., Tricin-lignins: Occurrence and quantitation of tricin in relation to phylogeny. Plant J. 88, 1046-1057 (2016).
73. W. Lan et al., Tricin, a flavonoid monomer in monocot lignification. Plant Physiol. 167, 1284-U1265 (2015).
74. W. Lan et al., Maize tricin-oligolignol metabolites and their implications for monocot lignification. Plant Physiol. 171, 810-820 (2016).
75. H.-M. Chang, E. B. Cowling, W. Brown, E. Adler, G. Miksche, Comparative studies on cellulolytic enzyme lignin and milled wood lignin of sweetgum and spruce. Holzforschung 29, 153-159 (1975).
76. H. Kim, J. Ralph, Solution-state 2D NMR of ball-milled plant cell wall gels in DMSO-d₆/pyridine-d₅. Org Biomol Chem 8, 576-591 (2010).
77. S. D. Mansfield, H. Kim, F. Lu, J. Ralph, Whole plant cell wall characterization using solution-state 2D-NMR. Nature protocols 7, 1579-1589 (2012).
78. H. Kim et al., Monolignol benzoates incorporate into the lignin of transgenic Populus trichocarpa depleted in C3H and C4H. ACS Sustain Chem Eng 8, 3644-3654 (2020).
79. J. Rencoret et al., Structural characterization of lignin isolated from coconut (Cocos nucifera) coir fibers. Journal of Agricultural and Food Chemistry 61, 2434-2445 (2013).
80. W. Lan et al., Elucidating tricin-lignin structures: Assigning correlations in HSQC spectra of monocot lignins. Polymers (Basel) 10, 916 (2018).
81. H. Zhang et al., dbCAN2: a meta server for automated carbohydrate-active enzyme annotation. Nucleic Acids Research 46, W95-W101 (2018).
82. R. D. Finn, J. Clements, S. R. Eddy, HMMER web server: interactive sequence similarity searching. Nucleic Acids Research 39, W29-W37 (2011).
83. R. C. Team (2019) R: A Language and Environment for Statistical Computing. (Vienna, Austria).

1. An isolated nucleic acid molecule encoding a β-etherase polypeptide wherein said polypeptide comprises copper and further wherein the activity of said polypeptide is independent of NAD⁺ and/or glutathione.

2. The isolated nucleic acid molecule according to claim 1, wherein said isolated nucleic acid molecule comprises a nucleotide sequence selected from the group consisting of:
i) a nucleotide sequence as set forth in SEQ ID NO: 18, SEQ ID NO:17. SEQ ID NO: 19, SEQ ID NO: 20, SEQ ID NO: 21, SEQ ID NO: 22, SEQ ID NO: 23, SEQ ID NO: 24, or SEQ ID NO: 25;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridisation conditions to sequence set forth in SEQ ID NO: 18, SEQ ID NO: 17, SEQ ID NO: 19, SEQ ID NO: 20, SEQ ID NO: 21, SEQ ID NO: 22, SEQ ID NO: 23, SEQ ID NO:24 or SEQ ID NO: 25;
iv) a nucleotide sequence that encodes a polypeptide comprising an amino acid sequence set forth in SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33;
v) a nucleotide sequence that encodes a polypeptide comprising an amino acid sequence wherein said amino acid sequence is modified by addition, deletion or substitution of at least one amino acid residue as represented in iv) above and has β-etherase activity.

3-9. (canceled)

10. An isolated β-etherase polypeptide wherein said polypeptide comprises copper and further wherein the activity of said polypeptide is independent of NAD⁺ and/or glutathione.

11. The isolated polypeptide according to claim 10, wherein said isolated polypeptide is selected from the group consisting of:
i) a polypeptide comprising or consisting of an amino acid sequence as set forth in SEQ ID NO: 26, SEQ ID NO: 27, SEQ ID NO: 28, SEQ ID NO: 29, SEQ ID NO: 30, SEQ ID NO: 31. SEQ ID NO: 32 OR SEQ ID NO: 33;
ii) a modified polypeptide comprising or consisting of a modified amino acid sequence wherein said polypeptide is modified by addition, deletion or substitution of at least one amino acid residue of the sequence set forth in SEQ ID NO: 26. SEQ ID NO: 27, SEQ ID NO: 28, SEQ ID NO: 29, SEQ ID NO: 30, SEQ ID NO: 31, SEQ ID NO: 32 or SEQ ID NO: 33, and which has β-etherase activity.

12-18. (canceled)

19. A vector comprising the nucleic acid molecule according to claim 1.

20. The vector according to claim 19. wherein the vector is an expression vector adapted for expression in a heterologous microbial host cell.

21. A cell transformed or transfected with the nucleic acid molecule according to claim 1.

22. The cell according to claim 21, wherein said cell is a heterologous host cell wherein said heterologous host cell does not naturally express the nucleic acid molecule.

23. The cell according to claim 21, wherein said cell is a bacterial cell, a fungal cell or a yeast cell.

24. (canceled)

25. The cell according to claim 23, wherein said fungal cell is an Aspergillus sp. cell. or wherein said fungal cell is not a Parascedosporium sp cell.

26. (canceled)

27. A composition comprising one or more polypeptides according to claim 10.

28. A composition according to claim 27, wherein said composition comprises at least the polypeptide set forth in SEQ ID NO: 9 or 26.

29. A composition according to claim 27, wherein said one more polypeptides are set forth in SEQ ID NO: 26, 27, 28, 29, 30, 31, 32 and 33.

30. A composition according to claim 27 wherein said composition further comprises one or more polypeptides for the saccharification of lignocellulose selected from the group consisting of cellulases, lytic polysaccharide monooxygenases, carbohydrate esterases, hemicellulases, glycosylhydrolases, endoglucanases, cellobiohydrolases, beta-glucosidases, xylanases, mannases, cellobiose dehydrogenases, and beta-xylosidases.

31. A method for the modification of plant biomass comprising the following steps:
i) contacting plant biomass with the composition according to
claim 27 to form a reaction mixture; and ii) incubating said reaction mixture under conditions which cleave β-ether linkages present in the plant biomass to obtain depolymerised lignin units.

32. The method according to claim 31, wherein;
said method comprises a further step of extracting said depolymerised lignin units from the reaction mixture;
said method comprises a further step of contacting said reaction mixture with a composition comprising one or more polypeptides for the saccharification of the processed lignocellulose; and/or
said method comprises extracting di- and/or monosaccharides_{.}

33. The method according to claim 31, wherein:
said depolymerised lignin units are selected from the group consisting of flavones and p-coumaric acid; and/or
said plant biomass is wheat straw or sugarcane bagasse.

34. The method according to claim 33 wherein said flavones are tricin.

35-36. (canceled)

37. The method according to claim 32, wherein said saccharification composition comprises or consist of one or more polypeptides selected from the group consisting of cellulases, lytic polysaccharide monooxygenases, carbohydrate esterases, hemicellulases, glycosylhydrolases, endoglucanases, cellobiohydrolases, beta-glucosidases, xylanases, mannases, cellobiose dehydrogenases, and beta-xylosidases.

38. (canceled)

39. A method for the manufacture of a β-etherase polypeptide comprising the following steps:
i) providing the cell according to claim 21 and cell culture medium,
ii) culturing the cell in i) above to express a β-eherase polypeptide wherein said polypeptide comprises copper and further wherein the activity of said polypeptide is independent of NAD⁺ and/or glutathione; and optionally,
iii) isolating said polypeptide from the cell or cell culture medium.

40. The method according to claim 39 wherein said polypeptide is isolated under denaturing conditions.
